SEQUENCE LISTING (42 sequences)

SEQ ID NO: 1 - DNA, 2103 bp, Homo sapiens
  misc_feature (1)..(2103): human calpain-2
atggcgggcatcgcggccaagctggcgaaggaccgggaggcggccgaggggctgggctcccacgagagggccatcaagtacctcaaccaggactacgaggcgctgcggaacgagtgcctggaggccgggacgctcttccaggacccgtccttcccggccatcccctcggccctgggcttcaaggagttggggccctactccagcaaaacccggggcatcgagtggaagcgccccacggagatctgcgctgacccccagtttatcattggaggagccacccgcacagacatctgccaaggagccctgggtgactgctggctgctggcagccattgcctccctcaccttgaatgaagaaatcctggctcgagtcgtccccctaaaccagagcttccaggaaaactatgcagggatctttcacttccagttctggcaatacggcgagtgggtggaggtggtggtggatgacaggctgcccaccaaggacggggagctgctctttgtgcattcagccgaagggagcgagttctggagcgccctgctggagaaggcatacgccaagatcaacggatgctatgaagcgctatcagggggtgccaccactgagggcttcgaagacttcaccggaggcattgctgagtggtatgagttgaagaagccccctcccaacctgttcaagatcatccagaaagctctgcaaaaaggctctctccttggctgctccatcgacatcaccagcgccgcggactcggaggccatcacgtttcagaagctggtgaaggggcacgcgtactcggtcaccggagccgaggaggttgaaagtaacggaagcctacagaaactgatccgcatccgaaatccctggggagaagtggagtggacagggcggtggaatgacaactgcccaagctggaacactatagacccagaggagagggaaaggctgaccagacggcatgaagatggagaattctggatgtctttcagtgacttcctgaggcactattcccgcctggagatctgtaacctgaccccagacactctcaccagcgatacctacaagaagtggaaactcaccaaaatggatgggaactggaggcggggctccaccgcgggaggttgcaggaactacccgaacacattctggatgaaccctcagtacctgatcaagctggaggaggaggatgaggacgaggaggatggggagagcggctgcaccttcctggtggggctcattcagaagcaccgacggcggcagaggaagatgggcgaggacatgcacaccatcggctttggcatctatgaggttccagaggagttaagtgggcagaccaacatccacctcagcaaaaacttcttcctgacgaatcgcgccagggagcgctcagacaccttcatcaacctccgggaggtgctcaaccgcttcaagctgccgccaggagagtacattctcgtgccttccaccttcgaacccaacaaggatggggatttctgcatccgggtcttttctgaaaagaaagctgactaccaagctgtcgatgatgaaatcgaggccaatcttgaagagttcgacatcagcgaggatgacattgatgatggattcaggagactgtttgcccagttggcaggagaggatgcggagatctctgcctttgagctgcagaccatcctgagaagggttctagcaaagcgccaagatatcaagtcagatggcttcagcatcgagacatgcaaaattatggttgacatgctagattcggacgggagtggcaagctggggctgaaggagttctacattctctggacgaagattcaaaaataccaaaaaatttaccgagaaatcgacgttgacaggtctggtaccatgaattcctatgaaatgcggaaggcattagaagaagcaggtttcaagatgccctgtcaactccaccaagtcatcgttgctcggtttgcagatgaccagctcatcatcgattttgataattttgttcggtgtttggttcggctggaaacgctattcaagatatttaagcagctggatcccgagaatactggaacaatagagctcgaccttatctcttggctctgtttctcagtactttga

SEQ ID NO: 2 - protein, 700 aa, Homo sapiens
  MISC_FEATURE (1)..(700): human calpain-2
MAGIAAKLAKDREAAEGLGSHERAIKYLNQDYEALRNECLEAGTLFQDPSFPAIPSALGFKELGPYSSKTRGIEWKRPTEICADPQFIIGGATRTDICQGALGDCWLLAAIASLTLNEEILARVVPLNQSFQENYAGIFHFQFWQYGEWVEVVVDDRLPTKDGELLFVHSAEGSEFWSALLEKAYAKINGCYEALSGGATTEGFEDFTGGIAEWYELKKPPPNLFKIIQKALQKGSLLGCSIDITSAADSEAITFQKLVKGHAYSVTGAEEVESNGSLQKLIRIRNPWGEVEWTGRWNDNCPSWNTIDPEERERLTRRHEDGEFWMSFSDFLRHYSRLEICNLTPDTLTSDTYKKWKLTKMDGNWRRGSTAGGCRNYPNTFWMNPQYLIKLEEEDEDEEDGESGCTFLVGLIQKHRRRQRKMGEDMHTIGFGIYEVPEELSGQTNIHLSKNFFLTNRARERSDTFINLREVLNRFKLPPGEYILVPSTFEPNKDGDFCIRVFSEKKADYQAVDDEIEANLEEFDISEDDIDDGFRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 3 - DNA, 2196 bp, Homo sapiens
  misc_feature (1)..(2196): human golgin-84
atgtcttggtttgttgatcttgctggaaaggcagaagatcttttaaaccgagttgatcaaggggctgcaacagctctcagtaggaaagacaatgccagcaacatatatagcaaaaatactgactatactgaacttcaccagcaaaatacagatttgatatatcagactggacctaaatctacgtatatttcatcagcagctgataacattcgaaatcaaaaagccaccatcttagctggcactgcaaatgtgaaagtaggatctcggacaccagtagaggcctctcatcctgttgaaaatgcatctgttcctaggccttcatcccattttgtgcgaagaaaaaagtcagaacctgatgatgagctgctgtttgattttcttaatagttcacagaaggagcctaccgggagggtggaaatcagaaaggaaaaaggcaagacacctgtctttcagagctctcagacatcaagtgtcagttctgtgaaccccagtgtaaccaccatcaaaaccattgaagaaaattcttttgggagccaaacccacgaagctgccagtaactcagattctagccatgaaggtcaagaggaatcttcaaaggaaaatgtgtcatcaaatgctgcctgccctgaccacaccccaacacctaatgatgatggcaaatcacatgaactgtctaaccttcgactggagaatcagctgctgaggaatgaagttcagtctttaaatcaagaaatggcctcgttactccaaagatccaaagagactcaagaagaattaaacaaagcaagagcaagagttgaaaagtggaatgctgaccattcaaagagtgatcgaatgactcgaggactccgagcccaagtagatgacctgactgaagctgtggctgcaaaggattcccagctggctgtactgaaagtgagactccaggaagctgaccagctactgagtactcgcacagaagcattagaagccttacagagtgaaaaatcacgaataatgcaggatcaaagtgaaggtaacagcctgcagaatcaagctctgcagacttttcaggagagactgcatgaagcggatgccactctgaagagagagcaggagagctataaacagatgcagagcgagtttgctgcacgccttaataaagtggaaatggaacgtcagaatttagcagaagcaattacactggccgaaagaaaatactcagatgagaagaagagggttgatgaactgcagcagcaagtcaagctgtataagttgaacttggagtcctctaagcaggaattaattgactacaagcaaaaagctactagaatactgcaatctaaggaaaaattgattaacagcttgaaagaaggctctggttttgaaggcctagatagcagcactgccagtagcatggagctggaagaacttcggcatgagaaagagatgcagagggaggaaatacagaagctgatgggccagatacatcagctcagatccgaattacaggatatggaggcacagcaagttaatgaagcagaatcagcaagagaacagttacaggatctgcatgaccaaatagctgggcagaaagcatccaaacaagaactagagacagaactggagcgactgaagcaggagttccactatatagaagaagatctttatcgaacaaagaacacattgcaaagcagaattaaagatcgagacgaagaaattcaaaaactcaggaatcagcttaccaataaaactttaagcaatagcagtcagtctgagttagaaaatcgactccatcagctaacagagactctcatccagaaacagaccatgctggagagtctcagcacagaaaagaactccctggtctttcaactggagcgcctcgaacagcagatgaactccgcctctggaagtagtagtaatgggtcttcgattaatatgtctggaattgacaatggtgaaggcactcgtctgcgaaatgttcctgttctttttaatgacacagaaactaatctggcaggaatgtacggaaaagttcgcaaagctgctagttcaattgatcagtttagtattcgcctgggaatttttctccgaagataccccatagcgcgagtttttgtaattatatatatggctttgcttcacctctgggtcatgattgttctgttgacttacacaccagaaatgcaccacgaccaaccatatggcaaatga

SEQ ID NO: 4 - protein, 731 aa, Homo sapiens
  MISC_FEATURE (1)..(731): human golgin-84
MSWFVDLAGKAEDLLNRVDQGAATALSRKDNASNIYSKNTDYTELHQQNTDLIYQTGPKSTYISSAADNIRNQKATILAGTANVKVGSRTPVEASHPVENASVPRPSSHFVRRKKSEPDDELLFDFLNSSQKEPTGRVEIRKEKGKTPVFQSSQTSSVSSVNPSVTTIKTIEENSFGSQTHEAASNSDSSHEGQEESSKENVSSNAACPDHTPTPNDDGKSHELSNLRLENQLLRNEVQSLNQEMASLLQRSKETQEELNKARARVEKWNADHSKSDRMTRGLRAQVDDLTEAVAAKDSQLAVLKVRLQEADQLLSTRTEALEALQSEKSRIMQDQSEGNSLQNQALQTFQERLHEADATLKREQESYKQMQSEFAARLNKVEMERQNLAEAITLAERKYSDEKKRVDELQQQVKLYKLNLESSKQELIDYKQKATRILQSKEKLINSLKEGSGFEGLDSSTASSMELEELRHEKEMQREEIQKLMGQIHQLRSELQDMEAQQVNEAESAREQLQDLHDQIAGQKASKQELETELERLKQEFHYIEEDLYRTKNTLQSRIKDRDEEIQKLRNQLTNKTLSNSSQSELENRLHQLTETLIQKQTMLESLSTEKNSLVFQLERLEQQMNSASGSSSNGSSINMSGIDNGEGTRLRNVPVLFNDTETNLAGMYGKVRKAASSIDQFSIRLGIFLRRYPIARVFVIIYMALLHLWVMIVLLTYTPEMHHDQPYGK

SEQ ID NO: 5 - protein, 27 aa, Homo sapiens
  MISC_FEATURE (1)..(27): Ac-Calpastatin (184-210)
DPMSSTYIEELGKREVTIPPKYRELLA

SEQ ID NO: 6 - protein, 27 aa, Homo sapiens
  MISC_FEATURE (1)..(27): Calpain inhibitor peptide (C-9181)
DPMSSTYIEELGKREVTIPPKTRELLA

SEQ ID NO: 7 - protein, 18 aa, Homo sapiens
  MISC_FEATURE (1)..(18): Calpastatin Related Peptide (SP007)
TYIEELGKREVTIPPKYR

SEQ ID NO: 8 - protein, 4 aa, Artificial
  caspase inhibitor Z-WEHD-FMK
WEHD

SEQ ID NO: 9 - protein, 4 aa, Artificial
  caspase inhibitor Z-DEVD-FMK
DEVD

SEQ ID NO: 10 - RNA, 21 nt, Artificial
  target sequence luciferase targeting siRNA
aacuuacgcugaguacuucga

SEQ ID NO: 11 - DNA, 21 bp, Artificial
  golgin-84 siRNA
ctgagtttagtggtcctaata

SEQ ID NO: 12 - DNA, 21 bp, Artificial
  GM130 siRNA
caggctggagttatacaagaa

SEQ ID NO: 13 - DNA, 21 bp, Artificial
  golgin-245 siRNA
caggaaatacatgaaatccaa

SEQ ID NO: 14 - DNA, 21 bp, Artificial
  giantin siRNA
aacttcatgcgaaggccaaat

SEQ ID NO: 15 - DNA, 21 bp, Artificial
  Gpp130 siRNA
caggaggacaatgttgatgaa

SEQ ID NO: 16 - DNA, 21 bp, Artificial
  CASP siRNA
cagcgcctgcacgatattgaa

SEQ ID NO: 17 - DNA, 23 bp, Artificial
  GAPDH forward primer
ggtatcgtggaaggactcatgac

SEQ ID NO: 18 - DNA, 23 bp, Artificial
  GAPDH reverse primer
atgccagtgagcttcccgttcag

SEQ ID NO: 19 - DNA, 19 bp, Artificial
  golgin-84 forward primer
aatgcaccacgaccaacca

SEQ ID NO: 20 - DNA, 19 bp, Artificial
  golgin-84 reverse primer
aggcaattggccttcttgc

SEQ ID NO: 21 - DNA, 23 bp, Artificial
  GM130 forward primer
aatatcagcagaggaatagccct

SEQ ID NO: 22 - DNA, 21 bp, Artificial
  GM130 reverse primer
cagcattgtccttgggtgtat

SEQ ID NO: 23 - DNA, 23 bp, Artificial
  golgin-245 forward primer
atgtatatgcaacaactgtgggg

SEQ ID NO: 24 - DNA, 22 bp, Artificial
  golgin-245 reverse primer
cgaggtgaagtaaacatcagcc

SEQ ID NO: 25 - DNA, 23 bp, Artificial
  giantin forward primer
ccctagaccctgaattacaccaa

SEQ ID NO: 26 - DNA, 20 bp, Artificial
  giantin reverse primer
ggcagaacagtccctccttg

SEQ ID NO: 27 - DNA, 19 bp, Artificial
  Gpp130 forward primer
ccctctccgcccagttaca

SEQ ID NO: 28 - DNA, 20 bp, Artificial
  Gpp130 reverse primer
ctcctcgtgttggcttttca

SEQ ID NO: 29 - DNA, 21 bp, Artificial
  CASP forward primer
aaagaccagcctgaaagtcgg

SEQ ID NO: 30 - DNA, 21 bp, Artificial
  CASP reverse primer
ccagggatgagctgaaaaagt

SEQ ID NO: 31 - RNA, 21 nt, Homo sapiens
  3′UTR (1)..(21): golgin-84 3`-UTR partial sequence
gagaacagugcacaagauuau

SEQ ID NO: 32 - RNA, 21 nt, Artificial
  firefly luciferase shRNA target sequence
aacuuacgcugaguacuucga

SEQ ID NO: 33 - DNA, 40 bp, Artificial
  golgin-84 primer (C-terminal myc-tag)
aaaaagcaggctgaaccatgtcttggtttgttgatcttgc

SEQ ID NO: 34 - DNA, 74 bp, Artificial
  golgin-84 primer (C-terminal myc-tag)
agaaagctgggtatcactacagatcttcttcagaaataagtttttgttctttgccatatggttggtcgtggtgc

SEQ ID NO: 35 - DNA, 41 bp, Artificial
  pDHE045 5-primer
aaaaagcaggctgaaccatgagtgtaaccaccatcaaaacc

SEQ ID NO: 36 - DNA, 46 bp, Artificial
  pDHE057 5-primer
aaaaagcaggctgaaccatgatcagaaaggaaaaaggcaagacacc

SEQ ID NO: 37 - DNA, 43 bp, Artificial
  pDHE058 5-primer
aaaaagcaggctgaaccatggtctttcagagctctcagacatc

SEQ ID NO: 38 - DNA, 41 bp, Artificial
  pDHE044 5-primer
aaaaagcaggctgaaccatgcaaggggctgcaacagctctc

SEQ ID NO: 39 - DNA, 44 bp, Artificial
  pDHE060 5-primer
aaaaagcaggctgaaccatgagttctgtgaaccccagtgtaacc

SEQ ID NO: 40 - DNA, 41 bp, Artificial
  pDHE105 5-primer
aaaaagcaggctgaaccatgggaaaatcacatgaactatct

SEQ ID NO: 41 - DNA, 34 bp, Artificial
  universal 3-primer
agaaagctgggtatcatttgccatatggttggtc

SEQ ID NO: 42 - protein, 13 aa, Homo sapiens
SVSSVNPSVTTIK

(19) United States
(12) Patent Application Publication
(10) Pub. No.: US 2011/0245150 A1
(43) Pub. Date: Oct. 6, 2011

(54) TARGETS FOR TREATMENT OF CHLAMYDIAL INFECTIONS

(75) Inventors: Dagmar Heuer, Berlin (DE); Thomas F. Meyer, Falkensee (DE); Anette Rejman Lipinski, Berlin (DE)

(73) Assignee: Max-Planck-Gesellschaft zur Foerderung der Wissenschaften e.V., Muenchen (DE)

(21) Appl. No.: 13/132,980

(22) PCT Filed: Dec. 4, 2009
(86) PCT No.: PCT/EP09/66435

§ 371 Date: Jun. 6, 2011

(30) Foreign Application Priority Data
Dec. 5, 2008 (EP) 08021174.1

(51) Int. Cl. A61K 38/05; C12Q 1/37; A61P 31/04; A61K 31/7088; A61K 38/06; A61K 38/07

(52) U.S. Cl. 514/2.4; 435/23; 514/20.2; 514/44 A

(57) ABSTRACT

The present invention relates to a screening method for identification of a compound suitable for the prevention, treatment or/and diagnosis of an infection with Chlamydiaceae.

The present invention relates to a screening method for identification of a compound suitable for the prevention, treatment or/and diagnosis of an infection with Chlamydiaceae.

The obligate intracellular bacterium Chlamydia trachomatis survives and replicates within a membrane bound vacuole, termed the inclusion, which intercepts host exocytic pathways to obtain nutrients¹⁻³. Like many other intracellular pathogens, C. trachomatis exhibits a marked requirement for host cell lipids, such as sphingolipids and cholesterol, produced in the endoplasmic reticulum (ER) and the Golgi apparatus (GA)⁴⁻⁶. However, the mechanisms by which intracellular pathogens acquire host cell lipids are not well understood¹⁻³. In particular, no host cell protein responsible for trafficking of Golgi-derived lipids to the chlamydial inclusions has yet been identified.

In the present invention it is demonstrated that Chlamydia infection induces GA fragmentation to generate Golgi ministacks surrounding the bacterial inclusion. Ministack formation is triggered by the proteolytic cleavage of the Golgi matrix protein, golgin-84. Inhibition of golgin-84 truncation prevents Golgi fragmentation, causing a block in lipid acquisition and maturation of C. trachomatis. Golgi fragmentation via RNAi knockdown of distinct Golgi matrix (GM) proteins prior to infection enhanced bacterial maturation. The data of the present invention functionally connect bacteria-induced golgin-84 cleavage, Golgi ministack formation, lipid acquisition, and intracellular pathogen growth. The data further demonstrate that C. trachomatis subverts the structure and function of an entire host cell organelle for its own advantage.

Members of the family Chlamydiaceae are obligate intracellular pathogens that replicate within membrane-bound vacuoles known as inclusions. They are responsible for several major diseases either in animals or humans. Until recently, the family Chlamydiaceae was represented by only a single genus known as Chlamydia that composed of four species: Chlamydia trachomatis, Chlamydia pneumoniae, Chlamydia psittaci and Chlamydia pecorum (Kaltenboeck, Kousoulas et al., 1993). In 1999, the chlamydial taxonomy was revised and the Chlamydiaceae family has been split into two genera (Chlamydia and Chlamydophila) encompassing three (Chlamydia trachomatis, Chlamydia suis, Chlamydia muridarum) and six (Chlamydophila pneumoniae, Chlamydophila abortus, Chlamydophila psittaci, Chlamydophila pecorum, Chlamydophila felis, Chlamydophila caviae) species, respectively (Everett, Bush et al., 1999). For simplicity, all members of the family Chlamydiaceae are referred, here, to as chlamydiae.

Chlamydiae possess a unique developmental cycle. This cycle consists of infectious and noninfectious stages that exhibit unique morphological, biochemical, and biological properties. The infectious elementary body (EB), which is non-replicating and metabolically inactive, attaches to and enters host cells (Moulder, 1991). After host cell entry, the EB is localized to a phagosome, and the primary differentiation process is initiated. This developmental process involves the commencement of bacterial metabolism and the conversion of the EB to the intracellular replicating form of the organism, termed the reticulate body (RB). The RB multiples by binary fission for a period of 24 to 36 h. After multiple rounds of replication the RB undergoes a secondary differentiation process back to an infectious EB. At this late stage in development (40 to 72 h) the host cell lyses and releases mature EBs that then reinfect neighboring host cells (Moulder, 1991).

Two distinguishing characteristics of chlamydiae are its developmental cycle and predilection for causing a persistent (chronic or latent) infections (Moulder, 1991), during which the normal developmental cycle is altered, producing aberrant RB-like forms. Persistency can be established in vitro using several methods, including treatment with cytokines or antibiotics or by deprivation of certain nutrients, such as amino acids (Beatty, Byrne et al., 1994) and iron (Al-Younes, Rudel et al., 2001). Persistent infections produced can revert to normally growing organisms when the suppressor is s removed or nutrients are replaced (Allan and Pearce, 1983; Al-Younes, Rudel et al., 2001). In humans, acute chlamydial infections can progress to persistent infections, which may lead to a pathogenic process that leads to chronic diseases including blindness, pelvic inflammatory disease, ectopic pregnancy, tubal factor infertility, arthritis, Alzheimer's disease and atherosclerosis (Hammerschlag, 2002; Villareal, Whittum-Hudson et al., 2002; Stephens, 2003).

Although they have a similar unique developmental cycle, chlamydiae cause a variety of human and animal diseases. Chlamydia trachomatis, primarily a pathogen of humans, is one of the most common bacterial pathogens that primarily infects columnar epithelial cells of the ocular and genital mucosae, causing sexually transmitted and ocular diseases in humans. These diseases have a significant impact on human health worldwide, causing trachoma, the leading cause of preventable blindness, and sexually transmitted diseases (STD) that include tubal factor infertility, life-threatening ectopic pregnancy, and pelvic inflammatory disease that often result in involuntary sterility (Stephens, 2003). Chlamydial STDs are also risk factors in cervical squamous cell carcinoma (Anttila, Saikku et al., 2001) and HIV infection (Chesson and Pinkerton, 2000). Infants are at risk for chlamydial eye infection and pneumonia if they pass through an infected cervix (Stephens, 2003). Chlamydia trachomatis strains (or serovars) L1, L2 and L3 are the etiological agents of the sexually transmitted systemic syndrome Lymphogranuloma venereum (LGV). Serovars A to C are primarily the agents responsible for the endemic blinding trachoma, while serovars D to K are associated with STDs (Guaschino and De Seta, 2000).

Chlamydophila pneumoniae is an important cause of human respiratory tract diseases, such as pneumonia, pharyngitis, sinusitis, otitis, asthma, acute bronchitis (Grayston, Campbell et al., 1990), persistent cough, chronic obstructive pulmonary disease (COPD), flu-like syndrome (Blasi, Arosio et al., 1999) and lung carcinoma (Laurila, Anttila et al. 1997). In addition, this pathogen is correlated with other non-pulmonary diseases, such as erythema nodosum (Erntell, Ljunggren et al., 1989), Guillain-Barré syndrome (Haidl, Ivarsson et al., 1992), endocarditis (Grayston, Campbell et al., 1990), Alzheimer's disease (Balin, Gerard et al., 1998), reactive arthritis (Villareal, Whittum-Hudson et al., 2002), meningoencephalitis (Koskiniemi, Gencay et al., 1996) and the blood vessel disease atherosclerosis (Campbell and Kuo, 2003).

Other species, such as C. psittaci, C. abortus and C. pecorum, are responsible for several major diseases in animals, mainly spontaneous abortion in livestock and systemic disease in birds, and can also infect rodents and cats (Schachter, 1999).

The biological mechanisms responsible for these differences in vertebrate host, tissue tropism and spectrum of diseases are unknown.

Serological surveys have shown that virtually every human has been infected with C. pneumoniae (Grayston, 2000). This prevalent pathogen causes 6 to 25% of community-acquired pneumonia. Further, this pathogen is also associated with other respiratory diseases as well as non-respiratory diseases, such as cancer and Alzheimer's disease. Importantly, increasing evidence demonstrated that C. pneumoniae is present and persistent at sites of arterial disease and, thus, contributes to coronary artery disease (Atherosclerosis). The presence of C. pneumoniae in atheromatous plaques has been demonstrated by several methods, such as polymerase chain reaction (PCR), immunocytochemistry, in situ hybridization, electron microscopy and by recovery of bacteria in tissue cultures (Ramirez, 1996). Furthermore, respiratory inoculation of C. pneumoniae in experimental animal models induced or accelerated the formation of atherosclerotic lesions (de Boer, van der Wal et al., 2000). This disease continues to be the principal cause of death in the U.S. and in most Western countries (Braunwald, 1997). For example, it causes nearly 25% of all deaths each year in the UK, whereas it causes about 40% of annual deaths in the U.S. (Gupta and Camm, 1998).

Chlamydia trachomatis causes sexually transmitted diseases (STDs) amounting to millions of cases per year and ocular infections that are the leading cause of preventable blindness in developing countries (Resnikoff et al. 2004, Gerbase, et al. 1998). As an obligate intracellular bacterium, it replicates inside the cell in a special niche, the membrane-bound inclusion. Recent studies reveal that many intracellular bacteria exploit the cellular secretory pathways from the endoplasmic reticulum (ER) through the Golgi apparatus (GA) to the plasma membrane for their own benefits at different stages (Salcedo et al., 2005). In this regard, intracellular bacteria such as Salmonella and Chlamydia spp. have been shown to localize to the GA (Grieshaber et al., 2002; Salcedo et al, 2003). Chlamydia spp. take up sphingolipids and cholesterol via BFA-sensitive transport from the GA, but the physiological significance of this interaction has not yet, been identified (Carabeo et al., 2003; Hackstadt et al., 1995; van Ooij et al., 2000). In addition, no host cell protein has been identified to date that is either transported from the GA into the chlamydial inclusion or is responsible for the Chlamydia-GA connections. Here we report the discovery of an unexpected link between intracellular chlamydial growth and a shift in Golgi morphology.

A subject of the present invention are screening methods for identifying a compound as being suitable for treatment, prevention or/and diagnosis of an infection with microorganisms from the family Chlamydiaceae, wherein the compound is capable of inhibiting the fragmentation of the Golgi apparatus or/and capable of inhibiting calpain, particularly capable of inhibiting calpain mediated cleavage of Golgi proteins.

A first preferred aspect of the present invention is a screening method for identification of a compound suitable for treatment, prevention or/and diagnosis of an infection with microorganisms from the family Chlamydiaceae, comprising identification of a calpain inhibitor.

In particular, this screening method comprises the steps

(a) providing calpain,

(b) contacting a compound with calpain,

(c) determining if the compound of (b) inhibits calpain activity, and

(d) selecting a compound which inhibits calpain activity.

It is preferred that calpain is provided in a cell or cell extract or as isolated protein.

The calpain inhibitor may act directly or may act indirectly. Direct inhibition includes an interaction of the inhibitor with calpain. Indirect interaction includes interaction of the inhibitor with a component in a cascade located upstream from calpain, so that the interaction causes inhibition of calpain. In particular, the interaction includes interaction with a component in a signalling cascade located upstream from calpain. The interaction may also include the interaction of calpain with a bacterial polypeptide, such as a bacterial protease. The bacterial polypeptide is in particular a chlamydial polypeptide.

It is also preferred that in this screening method, the calpain is calpain-2 and the compound suitable for treatment, prevention or/and diagnosis of an infection with microorganisms from the family Chlamydiaceae is a calpain-2 inhibitor.

In a second preferred aspect, the screening method for identification of a compound suitable for treatment, prevention or/and diagnosis of an infection with microorganisms from the family Chlamydiaceae comprises identification of a compound capable of inhibiting cleavage of a Golgi protein.

In particular, this screening method comprises the steps

(a) providing a Golgi protein,

(b) contacting a compound with a Golgi protein,

(c) determining if the compound of (b) inhibits Golgi protein cleavage, and

(d) selecting a compound which inhibits Golgi protein cleavage.

In this screening method, the Golgi protein is preferably selected from the group consisting of GASP, p115, GRASP55, GRASP65, golgin-45, Bicaudal D1 and D2, golgin-245, GMAp210, Rud3p, GM130, Gpp130, golgin-84, golgin-160, more preferably the Golgi protein is selected from the group consisting of Gpp130, golgin-84, golgin-160, giantin, and even more preferably the Golgi protein is golgin-84 or golgin-160.

It is also preferred that in this screening method, calpain cleavage of the Golgi protein is inhibited.

It is also preferred that in this screening method, the calpain is calpain-2 and the compound suitable for treatment, prevention or/and diagnosis of an infection with microorganisms from the family Chlamydiaceae is a calpain-2 inhibitor.

It is also preferred that in this screening method, the Golgi protein is provided in a cell or cell extract or as isolated protein. It is more preferred that the cell is infected with microorganisms from the family Chlamydiaceae.

In a third preferred aspect, the screening method for identification of a compound suitable for treatment, prevention or/and diagnosis of an infection with microorganisms from the family Chlamydiaceae comprises identification of a compound capable of inhibiting Golgi apparatus fragmentation in a cell infected with microorganisms from the family Chlamydiaceae.

In particular, this screening method comprises the steps
(a) providing a cell comprising a Golgi apparatus, which cell is capable of being infected with microorganisms from the family Chlamydiaceae,
(b) contacting the cell with a compound and with microorganisms from the family Chlamydiaceae,
(c) determining if the compound inhibits Golgi apparatus fragmentation in the cell, and
(d) selecting a compound which inhibits Golgi apparatus fragmentation in the cell.

In the present invention, fragmentation of the Golgi apparatus refers to dispersion of the Golgi apparatus into smaller vesicles. In the present invention, fragmentation may be completely (about 100%) dispersed into smaller vesicles, or partially (e.g. more than about 10%, more than about 20%, more than about 30%, more than about 40%, more than about 50%, more than about 60%, more than about 70%, more than about 80% or more than about 90%) dispersed into smaller vesicles.

It is also preferred that in this screening method, Golgi apparatus fragmentation depending upon cleavage of a Golgi protein is inhibited. It is more preferred that Golgi apparatus fragmentation depending upon calpain cleavage of a Golgi protein is inhibited.

It is also preferred that in this screening method, that the calpain is calpain-2 and the compound suitable for treatment, prevention or/and diagnosis of an infection with microorganisms from the family Chlamydiaceae is a calpain-2 inhibitor.

It is also preferred that in this screening method, the Golgi protein is selected from the group consisting of CASP, p115, GRASP55, GRASP65, golgin-45, Bicaudal D1 and D2, golgin-245, GMAp210, Rud3p, GM130, Gpp130, golgin-84, golgin-160, more preferably the Golgi protein is selected from the group consisting of Gpp130, golgin-84, golgin-160, giantin, and even more preferably the Golgi protein is golgin-84 or golgin-160.

The screening method of the present invention including the preferred aspects thereof may comprise a molecular assay or/and a cellular assay. In a cellular assay, the screening method of the present invention may comprise determining the intracellular propagation of Chlamydiaceae and selecting a compound which reduces intracellular propagation of Chlamydiaceae.

The screening method of the present invention including the preferred aspects thereof may comprise an in vitro or/and in vivo assay. An in vivo assay may comprise testing in an animal, such as mouse, rat, rabbit, etc.

In the screening method of the present invention including the preferred aspects thereof, calpain-mediated Golgi fragmentation during chlamydial infection may involve the regulation of lipid synthesis.

In the screening methods of the present invention, calpain inhibition, calpain-mediated cleavage of a Golgi protein or/and Golgi fragmentation may be detected by detection of cleavage within amino acids 148-158 of golgin-84, in particular at amino acid S157 of golgin 84. Calpain inhibition, calpain-mediated cleavage of a Golgi protein or/and Golgi fragmentation may also be detected by detection of a 65 kDa fragment of golgin-84. Calpain inhibition, calpain-mediated cleavage of a Golgi protein or/and Golgi fragmentation may also be detected by inhibition of Golgi ministack formation around the chlamydial inclusion. Calpain inhibition, calpain-mediated cleavage of a Golgi protein or/and Golgi fragmentation may also be detected by alteration of lipid synthesis including sphingolipid synthesis. Calpain inhibition, calpain-mediated cleavage of a Golgi protein or/and Golgi fragmentation may also be detected by alteration of transport of lipids to the chlamydial inclusion, for instance alteration of transport of sphingolipids. The sphingolipid may be a ceramide, or may contain a ceramide. In particular, increased transport of sphingolipids to the chlamydial inclusion, for instance at later time points during the infection, indicates an increased Golgi fragmentation, or decreased transport of sphingolipids to the chlamydial inclusion, for instance at later time points during the infection, indicates a decreased Golgi fragmentation. Further read-out parameters or/and control parameters of calpain inhibition, calpain mediated cleavage or/and Golgi fragmentation suitable in the screening methods, of the present invention can be found in the Examples.

In the screening method of the present invention, an inhibitor of a protease may be identified which inhibits cleavage of a Golgi protein or inhibits Golgi apparatus fragmentation, as described herein. The protease may be selected from calpains and caspases, such as calpain-2 and caspase-1. In particular, the protease is selected from calpains. More particular, the protease is calpain-2.

Yet another aspect of the present invention is a pharmaceutical composition comprising as an active ingredient an inhibitor of calpain, an inhibitor of cleavage of a Golgi protein, or/and an inhibitor of Golgi apparatus fragmentation, optionally together with suitable carriers, diluents, adjuvants or/and auxiliary substances.

The pharmaceutical composition may also comprise an inhibitor of a protease as indicated herein.

In particular, the pharmaceutical composition of the present invention comprises an inhibitor of calpain, which preferably is capable of inhibiting cleavage of a Golgi protein as described herein or/and capable of inhibiting Golgi apparatus fragmentation.

The pharmaceutical composition of the present invention is preferably for use in medicine, such as in veterinary medicine or in human medicine. The pharmaceutical composition of the present invention is particularly suitable for the treatment, prevention or/and diagnosis of an infection with microorganisms from the family Chlamydiaceae.

The pharmaceutical composition of the present invention may comprise at least one further active ingredient for the prevention or/and treatment of infections with microorganisms from, the family Chlamydiaceae, such as an antibiotic. The constituents of this combination may exhibit a synergistic effect in the treatment of a Chlamydiaceae infection.

In the pharmaceutical composition of the present invention the at least one further active ingredient may be selected from antibiotics such as macrolides, quinolones and combinations thereof. This combination may be useful in an infection with Chlamydiaceae, wherein the antibiotic is not sufficiently successful when taken alone to treat the Chlamydiaceae infection.

The pharmaceutical composition of the present invention may be suitable for the treatment of patients with chronic infections with microorganisms from the family Chlamydiaceae.

In the pharmaceutical composition of the present invention, the calpain inhibitor is preferably a calpain-2 inhibitor.

The pharmaceutical composition may be administered, by any known method, such as by ingestion (e.g. tablets, troches, syrups, etc.), or by injection (e.g. parenteral, subcutaneous or intravenous routes).

Yet another aspect of the present invention is the use of a pharmaceutical composition of the present invention or an inhibitor of calpain, an inhibitor of cleavage of a Golgi protein, or/and an inhibitor of Golgi apparatus fragmentation, for the manufacture of a medicament for treating, preventing or/and diagnosing of an infection with microorganisms from the family Chlamydiaceae.

An inhibitor of a protease as indicated herein may also be used for the manufacture of a medicament for treating, preventing or/and diagnosing of an infection with microorganisms from the family Chlamydiaceae.

Yet another aspect of the present invention is a method for treatment or/and prevention of an infection with microorganisms from the family Chlamydiaceae, the method comprising the administration of an inhibitor of calpain, an inhibitor of cleavage of a Golgi protein, or/and an inhibitor of Golgi apparatus fragmentation, in a amount effective in therapy or/and prevention to a subject in need thereof.

The method may also comprise the administration of an inhibitor of a protease as indicated herein.

In the method for treatment or/and prevention of the present invention, an inhibitor known in the art of calpain, of Golgi protein cleavage, or/and of Golgi apparatus fragmentation may be administered.

The method of the present invention for treatment or/and prevention may further comprise administering at least one further active ingredient for the prevention or/and treatment of an infection with microorganisms from the family Chlamydiaceae in a amount effective in therapy or/and prevention.

In the method of the present invention for treatment or/and prevention the calpain inhibitor is preferably a calpain-2 inhibitor.

Yet another aspect of the present invention is a method of diagnosing an infection with a microorganism from the family Chlamydiaceae, comprising determination of a Golgi protein, Golgi apparatus fragmentation or/and calpain in a biological sample. The biological sample may be a clinical sample such as a cell, a body fluid or a tissue obtained from a subject suspected to suffer from a chlamydial infection.

The method of diagnosing may also comprise determination of a protease as indicated herein.

In the diagnosis method of the present invention, the calpain may be calpain-2.

The cell which may be employed in any of the screening methods of the present invention may be a cell capable of being infected with microorganisms from the family Chlamydiaceae. The cell may be a eukaryotic cell, such as a human cell, a mouse cell, a rat cell. The cell may be a freshly isolated cell, a cell in primary cell culture, or a cell line. An example of a suitable cell line is HeLa.

In the present invention, calpain may be any known calpain protein. The calpain may be selected from human and non-human calpains. The calpain is preferably a mammalian calpain and may e.g. comprise
(a) the amino acid sequence of SEQ. ID. NO:2,
(b) an amino acid sequence which is at least 70%, preferably at least 80%, more preferably at least 90%, even more preferably at least 95%, most preferably at least 99% identical to the sequence of (a), or/and
(c) a fragment of the sequences of (a) or (b).

SEQ ID NO:2 is the amino acid sequence of calpain-2. SEQ ID NO:1 is a nucleic acid sequence encoding calpain-2

The calpain as employed herein may e.g. be encoded by a nucleic acid comprising
(a) the nucleotide sequence of SEQ ID NO:1,
(b) a nucleotide sequence corresponding to the sequence of (a) within the scope of the degeneracy of the genetic code,
(c) a nucleotide sequence hybridizing with the sequence of (a) or/and (b) under stringent conditions,
(d) a nucleotide sequence which is at least 70%, preferably at least 80%, more preferably at least 90%, even more preferably at least 95%, most preferably at least 99% identical to the sequence of (a), (b) or/and (c)
(e) a fragment of the sequences of (a), (b), (c), or/and (d), or/and
(f) a sequence complementary to the sequences of (a), (b), (c), (d), or (e).

A Golgi protein as employed herein is a protein belonging to the golgin or GRASP (golgi reassembly stacking protein) family of Golgi stacking proteins (see e.g. Short et al., 2005), and may be selected from the group consisting of CASP, p115, GRASP55, GRASP65, golgin-45, Bicaudal D1 and D2, golgin-245, GMAp210, Rud3p, GM130, Gpp130, golgin-84, golgin-160 and giantin. A Golgi protein is preferably selected from Gpp130, golgin-84, golgin-160 and giantin. More preferably, the Golgi protein is golgin-84 or golgin-160.

The golgin-84 as employed herein is preferably a mammalian golgin-84 and may e.g. comprise
is (a) the amino acid sequence of SEQ ID NO:4,
(b) an amino acid sequence which is at least 70%, preferably at least 80%, more preferably at least 90%, even more preferably at least 95%, most preferably at least 99% identical to the sequence of (a), or/and
(c) a fragment of the sequences of (a) or (b).

The golgin-84 as employed herein may e.g. be encoded by a nucleic acid comprising
(a) the nucleotide sequence of SEQ ID NO:3,
(b) a nucleotide sequence corresponding to the sequence of (a) within the scope of the degeneracy of the genetic code,
(c) a nucleotide sequence hybridizing with the sequence of (a) or/and (b) under stringent conditions,
(d) a nucleotide sequence which is at least 70%, preferably at least 80%, more preferably at least 90%, even more preferably at least 95%, most preferably at least 99% identical to the sequence of (a), (b) or/and (c)
(e) a fragment of the sequences of (a), (b), (c), or/and (d), or/and
(f) a sequence complementary to the sequences of (a), (b), (c), (d), or (e).

A person skilled in the art is familiar with stringent hybridization conditions (see e. g. Sambrook J. et al., 1989, Molecular Cloning, A Laboratory Manual, Cold Spring Harbor Press, Plainview, N.Y.). Preferably, hybridization under stringent conditions means that, after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C., and most preferably at 68° C., a positive hybridization signal is observed.

Identity of sequences as indicated herein refers to the regions of maximum overlap of the sequences to be compared. Common algorithms, such as FASTA or BLAST, may be used for calculation of the percentage of identical positions of two sequences.

Polypeptide fragments, for instance of calpain or golgin-84, may have a length of at least 20, at least 50, at least 100, or at least 200 amino acid residues. Polypeptide fragments, for instance of calpain or golgin-84, may have a length of at the maximum of 300, at the maximum of 100, or at the maximum of 50 amino acid residues.

Nucleic acid fragments, for instance encoding fragments of calpain or golgin-84, may have a length of at least 60, at least 150, at least 300, or at least 600 nucleotide residues. Nucleic acid fragments, for instance encoding fragments of calpain or golgin-84, may have a length of at the maximum of 900, at the maximum of 300, or at the maximum of 150 nucleotide residues.

The microorganism from the family Chlamydiaceae as used herein may be a microorganism from the genus Chlamydia, in particular Chlamydia trachomatis, or may be from the genus Chlamydophila, in particular Chlamydophila pneumoniae.

The methods of the present invention may comprise recombinant expression of a polypeptide. Suitable methods for recombinant expression are known in the art. Where appropriate, any of the methods of the present invention may comprise the recombinant expression of calpain or/and a Golgi protein as defined herein.

The methods of the present invention may comprise overexpression of a polypeptide. Suitable method for overexpression are known in the art. Where appropriate, any of the methods of the present invention may comprise the overexpression of calpain or/and a Golgi protein as defined herein.

Specific inhibitors of calpain are known in the art, e.g. peptide inhibitors such as calpeptin and calpain inhibitor III. The calpain inhibitor of the present invention may be selected from the following known calpain inhibitors (in brackets: alias names or/and the chemical structure):
Calpain Inhibitor I (MG-101, Ac-Leu-Leu-Nle-CHO)
Calpain Inhibitor II (Ac-Leu-Leu-Met-CHO)
Calpain Inhibitor III (MDL-28170 Z-Val-Phe-CHO)
Calpain Inhibitor IV (Calpain Inhibitor 1, Z-Leu-Leu-Tyr-CH₂F)
Calpain Inhibitor IV-2 (Z-Leu-Leu-Leu-CHO)
Calpain Inhibitor V (Mu-Val-HPh-CH₂F)
Calpain Inhibitor VI (4-Fluorophenylsulfonyl-Val-Leu-CHO)
Calpain Inhibitor VII (Leu-Leu-Phe-CH₂Cl)
Calpain Inhibitor X (Z-L-Abu-CONH-ethyl)
Calpain Inhibitor XII (Z-Leu-Nva-CONH—CH₂-2-Pyridyl)
Calpain Inhibitor 2 (Mu-Phe-HPh-CH₂F)
Calpeptin (Z-Leu-Nle-CHO)
Z-Leu-Leu-Tyr-CHN2 (Z-Leu-Leu-Tyr-CHN₂)
Z-Leu-Tyr-CH2Cl (Z-Leu-Tyr-CH₂Cl)
Z-Phe-Tyr-CHO
Z-Leu-Leu-CHO
Leupeptin (Ac-Leu-Leu-L-Argininal)
E-64 ([L-trans-3-Carboxyoxirane-2-carbonyl]-L-Leu-agmatine)
E-64-c ([L-trans-3-Carboxyoxirane-2-carbonyl]-L-Leu-(3-methylbutyl)amide)
E-64-d (EST, Loxistatin, [L-trans-3-Ethoxycarbonyloxirane-2-carbonyl]-L-Leu-(3-Methylbutyl)amide)
PD150606 (3-(4-Iodophenyl)-2-mercapto-(Z)-2-propenoic acid)
PD151746 (3-(5-Fluoro-3-indolyl)-2-mercapto-(Z)-2-propenoic acid)
PD145305 (2-Mercapto-3-phenylpropanonic acid)
Calpastatin, domain 1 (134 aar)
Ac-Calpastatin (184-210) (Ac-DPMSSTYIEELGKREVTIPPKYRELLA-NH₂)
Calpain inhibitor peptide (C-9181, DPMSSTYIEELGKREVTIPPKTRELLA)
Calpastatin Related Peptide (SP007, TYIEELGKREVTIPPKYR)
AK265
AK275
AK295
CX295
BSF409425
N-(1-carbamoyl-1-oxohex-1-yl)-2-[E-2-(4-dimethyl-aminomethylphenyl)-ethen-1-yl]benzamide (5b)
A-705253 (CAL 9961)
Naphtalene 19c
A-705239
ATA (Aurintricarboxylic acid)
Quinolinecarboxamides
MDL-104903
IDDB20169 (Cephalon)
CEP-3122
208051-37-0 (Cephalon-3)
BN-82270
A-705253
CAT-811
SNT-198438
SNJ-1945
163039-88-1
EP-475
TH-3501,
   wherein Z is benzyloxycarbonyl, Mu is morphlinoureidyl, and HPh is homophenylalanyl. For example, the pharmaceutical composition of the present invention may comprise a calpain inhibitor selected from known calpain inhibitors as indicated herein. For example, in the method of the present invention for treatment or/and prevention of an infection with microorganisms from the family Chlamydiaceae, a calpain inhibitor selected from known calpain inhibitors as indicated herein may be administered. For example, a known calpain inhibitor as described herein may be used in the method of the present invention for diagnosing an infection with microorganisms from the family Chlamydiaceae. For example, a known calpain inhibitor as described herein may be used according to the present invention for the manufacture of a medicament for treating, preventing or/and diagnosing of an infection with microorganisms from the family Chlamydiaceae.

The inhibitor of the present invention, in particular of calpain, may also be a nucleic acid, e.g. an antisense molecule, a ribozyme or an RNA interference mediating molecule such as siRNA molecule. Based upon the sequences of interest, such as SEQ ID NOs: 1 or 2 of the present invention, such a siRNA molecule may be constructed by known methods.

The siRNA molecule as employed herein is preferably a double stranded RNA molecule comprising a partial sequence of the sequence of interest, such as SEQ ID NO:1, and having a length of the double-stranded region of 15 to 29, 17 to 25, 19 to 23, or 21 nucleotides. The siRNA molecule may comprise at least one single-stranded region, such as an overhang of 1, 2, 3, 4 or 5 or even more nucleotides at one or both ends of the double-stranded region.

The inhibitor of the present invention, in particular of calpain, may also be an antibody, e.g. a polyclonal, monoclonal, chimeric, humanised or human antibody.

The invention is illustrated by the following figures and examples.

### FIGURE LEGENDS

FIG. 1

C. trachomatis Infection Triggers Breakdown of GA into Ministacks.

(a) Timelapse microscopy of GFP-GM130 expressing HeLa cells infected with C. Trachomatis (MOI=2). Arrows indicate inclusions (b) Comparison of numbers and average size of GM130 signal in control, infected and staurosporine-treated cells. Error bars indicate mean±std. dev of three independent experiments. (c) Control and infected HeLa cells were stained with antibodies against GM130 or giantin (red channel) 28 h p.i. Chlamydia was detected using antibodies to LPS or Hsp60 (green channel). Scale bar, 10 μm. (d, e) Ultrastructural analysis of control (d) and cells infected with C. trachomatis for 24 h (e). Scale bar, 1 μm.

FIG. 2

Cleavage of Golgin-84 in Infected Cells is Associated with GA Fragmentation.

(a) Immunoblots of lysates (h p.i. indicated) showing golgin-84, GM130, giantin, cHsp60 and tubulin expression in infected (+) or mock-infected HeLa cells (−) (b) Immunoblots of lysates from C. trachomatis- (+) or mock-infected (−) HeLa cells (24 h p.i.), after addition of caspase inhibitors Z-WEHD-FMK (caspase-1/5 inhibitor), ZDEVD-FMK (caspase-3/7 inhibitor) or caspase inhibitor IV (pan-caspase inhibitor) at 9 h p.i. (c) Immunoblots of lysates from C. trachomatis- (+) or mock-infected (−) HeLa cells (26 h p.i.) treated with specific calpain (Calpeptin; CaIpIII), proteasome (MG-132; PS1) and cathepsin (CA-074) inhibitors at 8 h p.i. (d) Merge of GM130 (red channel) and LPS (blue channel) in untreated (control) or Z-WEHD-FMK-treated cells after infection. Scale bar, 10 μm. (e) C. trachomatis maturation in untreated control, WEHD-FMK-treated control and golgin-84 KD cells 48 h p.i. Numbers of infectious bacteria measured as IFU/ml are depicted in log scale. Error bars, mean+std.dev of duplicates. (f) GM130 immunostaining of infected golgin-84 KD cells stably expressing WT golgin-84 or the mutant (D218aa). Inclusions are seen as black holes in the GFP channel as GFP s does not cross the inclusion membrane. Scale bar, 10 μm (g) Replication of C. trachomatis in mutant (D218aa) cell lines at 48 h p.i. Numbers of infectious bacteria measured as total IFU/ml. Error bars, mean+std.dev of duplicates.

FIG. 3

RNAi-Mediated Fragmentation of the GA Enhances Chlamydial Propagation.

(a) GM130 immunostaining of HeLa cells transfected with siRNAs directed against the indicated golgins 4 days p.t. GM130 KD cells were immunostained for giantin to visualise the GA. Scale bar, 10 μm. (b) Numbers of infectious bacteria measured as IFU/ml at 48 h p.i. in various transient golgin KD cells (c) and in stable golgin-84 KD and control cell lines at 24 h and 40 h p.i. (in log scale). Representative experiments performed in duplicates shown. Error bars, mean+std.dev of duplicates. (d, e) TEM of C. trachomatis-infected (24 h p.i.). (d) stable golgin-84 KD (e) and control cells. Black arrowheads, infectious bacteria (EB); white arrowheads, membranous structures. A representative picture of a non-infectious RB and an infectious EB is depicted in the inlay. Scale bar, 2 μm.

FIG. 4

Functions of a Fragmented GA.

(a) Infected or uninfected cells were stained with GS-II Alexa Fluor 594 28 h p.i. Nuclei and bacteria were counterstained with Hoechst. Scale bar, 10 μm (b) HeLa cells were either left uninfected (no infection) or infected with C. trachomatis (MOI=2). One infected sample was treated with 80 μM Z-WEHD-FMK 9 h p.i. (infection+inhibitor). BODIPY FL C5-Ceramide transport in infected cells+/−inhibitor and control cells was analysed by time-lapse microscopy 30 min after addition of labelled ceramide. Nuclei and bacteria were counterstained with Hoechst. Scale bar, 10 μm; arrowheads, inclusions; arrows, GA.

FIG. 5

Plasmids (A) and corresponding cell lines (B) as employed herein, and which are suitable for use in the methods of the present invention.

FIG. 6

MS/MS-Spectrum of Golgin-84 Peptide (157-169)

Golgin-84 cleavage product (65 kDa fragment) was In-Gel digested with trypsin and analysed by Nano-LC-MS/MS. Nearly complete b- and y-series and a MASCOT ion score of 55 (15 above the identity threshold) prove the identity of this peptide. Identified fragments are annotated within the spectrum and marked on the sequence with filled circles.

FIG. 7

Nucleotide sequence of human calpain-2 (SEQ ID NO:1), amino acid sequence of human calpain-2 (SEQ ID NO:2), nucleotide sequence of human golgin-84 (SEQ ID NO:3), amino acid sequence of human golgin-84 (SEQ ID NO:4).

### EXAMPLE

In the GA newly synthesised proteins and lipids, including sphingolipids, are stepwise modified and then sorted to various locations inside, or are exported out of the cell⁷. The organisation of the GA is thought to depend on cytoplasmic structural proteins, including golgins, which form the GM⁸. Golgins belong to a large family of proteins with coiled-coil domains that localise to the GA⁹. Fragmentation of the GA is a common feature of a number of physiological processes such as mitosis and apoptosis. During mitosis, fragmentation is thought to be triggered by the phosphorylation of several proteins, including golgin-84, GM130 and GRASP-65 as well as the inactivation of small GTPases¹⁰⁻¹³. During apoptosis, fragmentation is induced by caspase-dependent cleavage of golgins, such as giantin, golgin-160 and p115¹⁴⁻¹⁶.

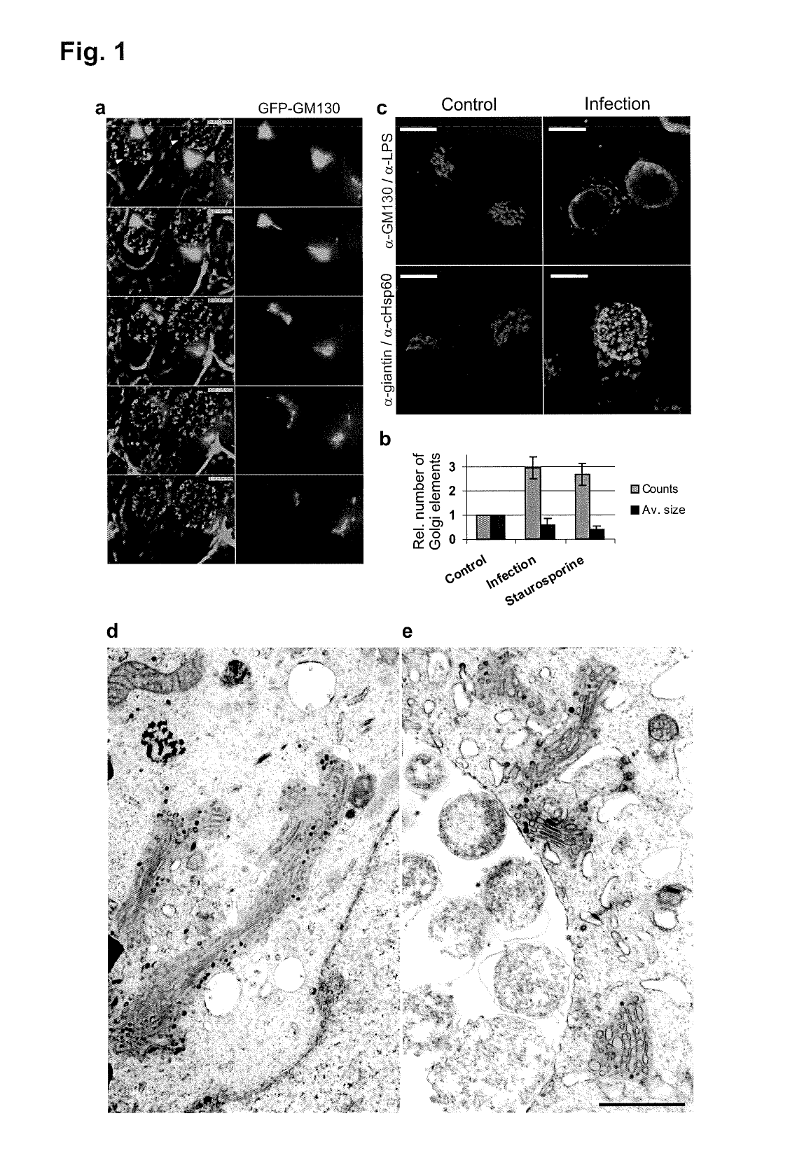

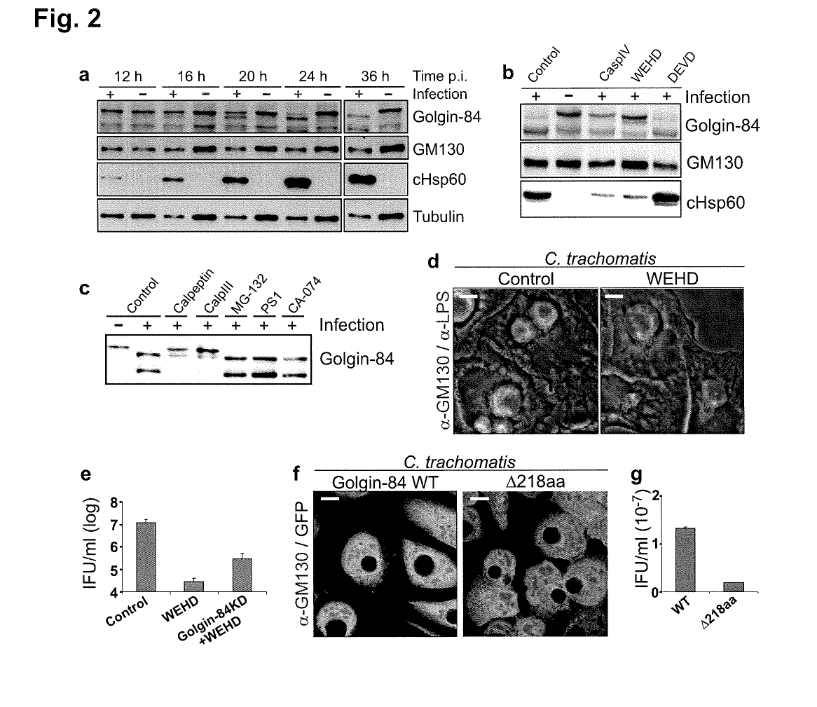

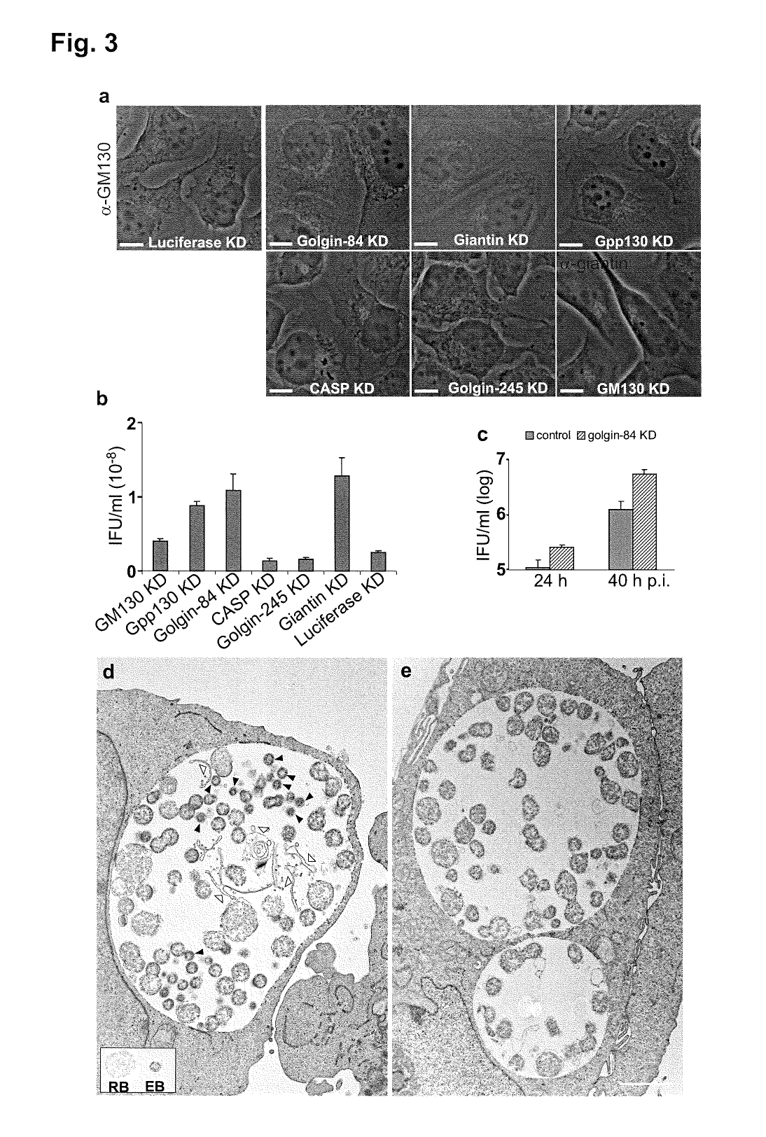

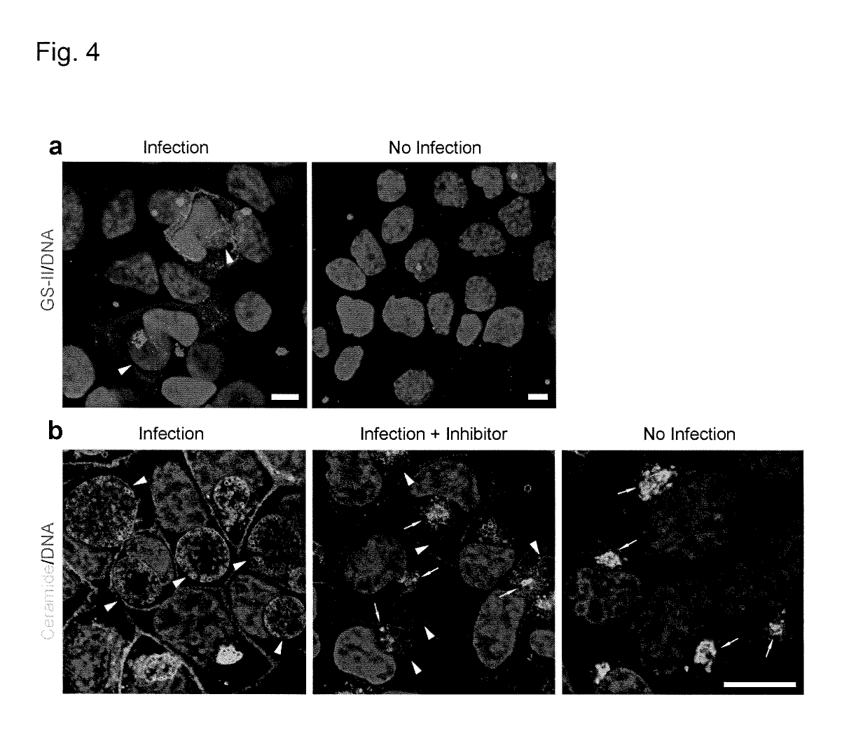

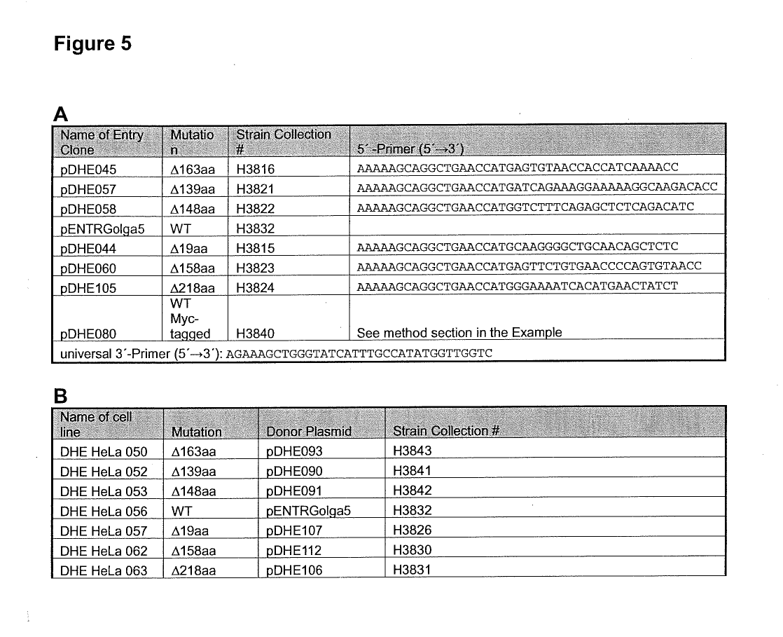

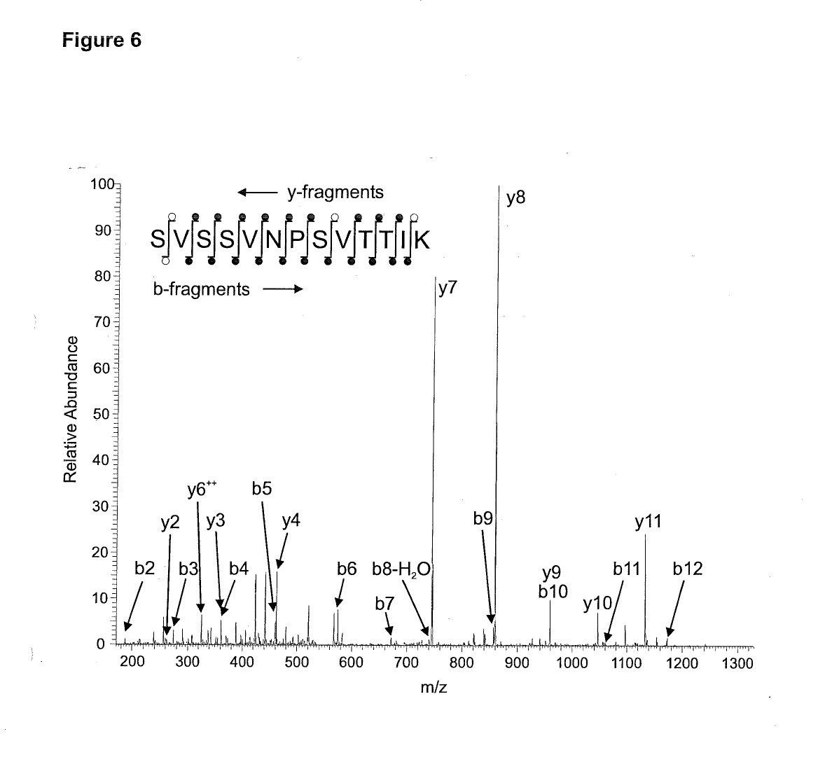

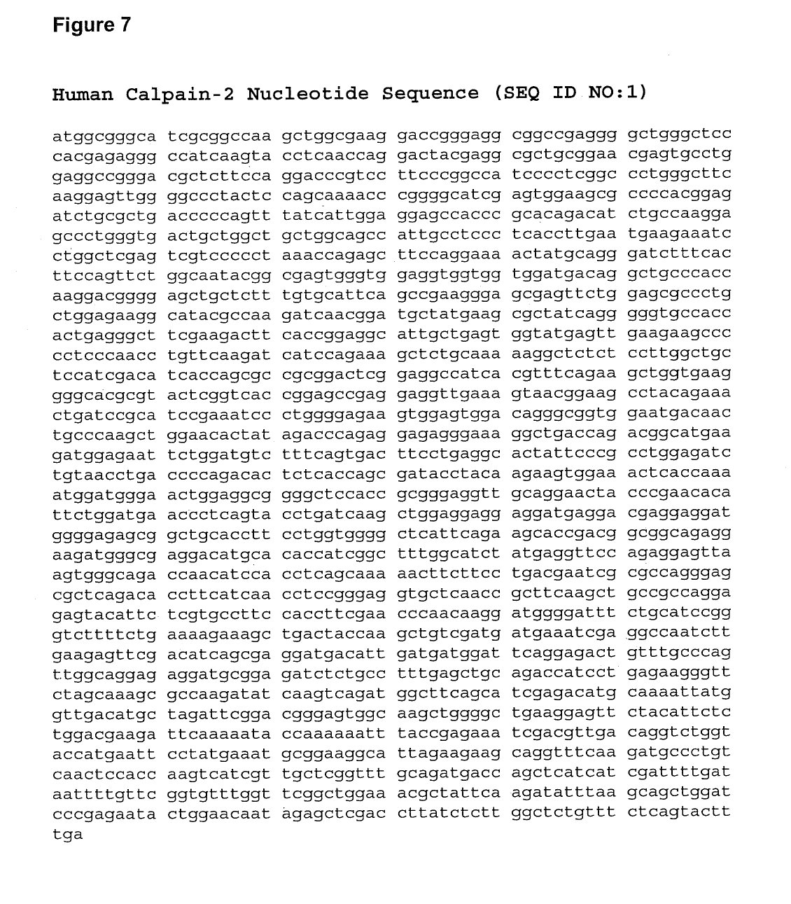

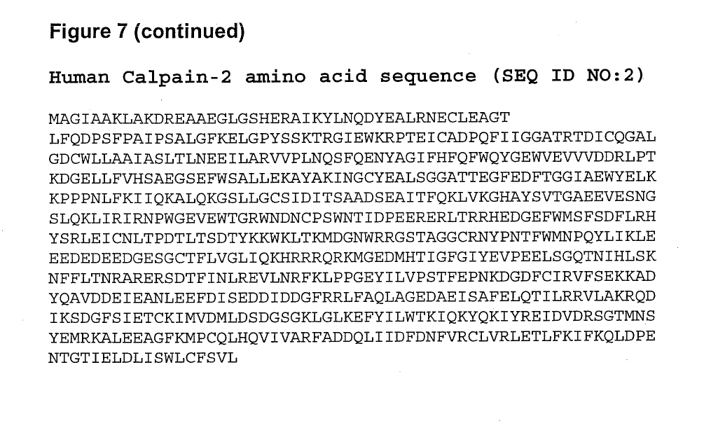

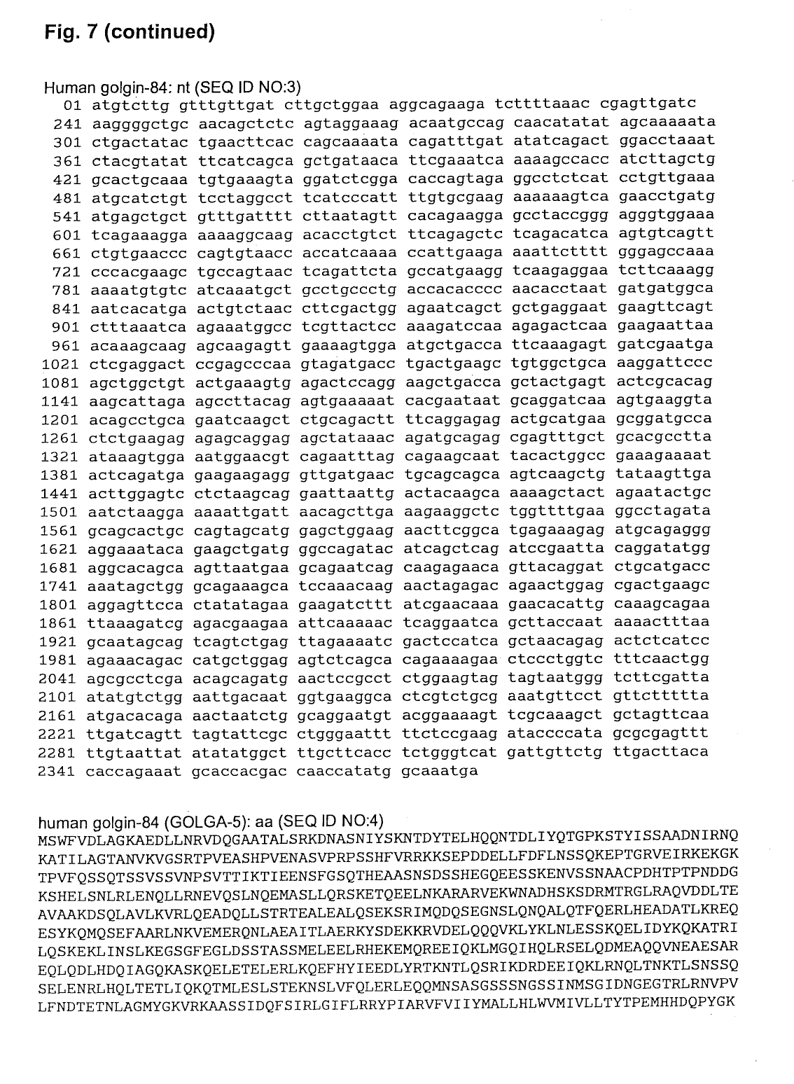

To investigate the mechanisms underlying sphingolipid trafficking to the chlamydial inclusion, we began by observing GA structure in C. trachomatis-infected epithelial cells. Live cell microscopy revealed a focused GFP-GM130 signal, typical of the normal ribbon-like structure of the GA, in close association with the bacterial inclusion at an early stage of infection (FIG. 1a). As infection proceeded the signal expanded, indicating a progressive dispersion of GFP-GM130-positive structures, resulting in the disassembly of the Golgi ribbon structure. The remaining smaller Golgi elements, albeit increased in number (FIG. 1b), were aligned along the inclusion membrane (FIG. 1a, c). Transmission electron microscopy (TEM) revealed a typical GA structure consisting of laterally-linked stacked cisternae extending over several micrometers in noninfected cells (FIG. 1d), whereas in infected cells a fragmented GA composed of relatively short Golgi stacks that were neither laterally-linked nor aligned to each other was observed (FIG. 1e). Our results indicate that C. trachomatis-infection induces fragmentation of the GA into small, albeit intact Golgi ministacks.

We then investigated the status of various golgins in infected cells. Immunoblotting of lysates revealed golgin-84 was sequentially processed during infection, yielding two distinct fragments of ˜78 kDa and ˜65 kDa; the smaller fragment accumulated at later time points p.i. (FIG. 2a). Cleavage was time and multiplicity of infection (MOI) dependent (FIG. 2a). Interestingly, golgin-84 cleavage also occurred in a range of epithelial cell lines infected with a variety of chlamydial strain. Thus, infection with Chlamydia species leads to stepwise processing of golgin-84, accompanied by GA fragmentation.

Cleavage of golgins has previously been associated with GA fragmentation during apoptosis₁₄₋₁₆. Although Chlamydia infected cells are largely protected from apoptosis^{17,18}, we investigated the possible involvement of caspases in golgin-84 cleavage. Treatment of infected cells with pan-caspase inhibitor IV and Z-WEHD-FMK, an inhibitor of inflammatory caspases, elicited a near complete blockage of C. trachomatis-induced cleavage of golgin-84. In contrast, cleavage was unaffected by the addition of the apoptotic caspase inhibitor Z-DEVD-FMK (FIG. 2b). Furthermore, stable expression of various N-terminally truncated versions of golgin-84 in golgin-84 knockdown (KD) cells, suggested the golgin-84 cleavage site leading to the 65 kDa fragment is most likely contained within amino acids 148-158. To determine the precise cleavage site generating the 65 KDa fragment, golgin-84 fused to myc-tag was transiently expressed in Chlamydia-infected cells. Tagged golgin-84 was precipitated from cells late in the infection cycle to assure is complete cleavage of golgin-84 into the predominant 65 kDa fragment. Mass spectroscopy (MS) analysis of isolated and trypsin-digested golgin-84 demonstrated a major proportion of golgin-84 was cleaved at amino acid S157 (FIG. 6). Insilico analysis of amino acids 155-157 (http://merops.sanger.ac.uk/) revealed calpain-2 as a candidate protease for golgin-84 cleavage. Calpains represent a group of intracellular cysteine proteases that locate to the cytosol, ER and GA^{19,20} and are considered as biomodulators₂₁. To test their presumptive role, infected cells were treated with several specific membrane-permeable inhibitors. Interestingly, both calpain inhibitors, calpeptin and calpain inhibitor III (CaIpIII), nearly completely blocked the generation of the 65 kDa golgin-84 fragment in infected cells, whereas formation of the 78 kDa fragment was less affected (FIG. 2c). Taken together, our data reveal that both inflammatory caspases and calpains are involved in the successive cleavage of golgin-84 and that the 65 kDa fragment of golgin-84 is likely generated by calpain cleavage at position S157.

We next assessed the interdependence between golgin-84 cleavage, Golgi fragmentation and chlamydial propagation. Golgin-84 cleavage was blocked via treatment of infected cells with Z-WEHD-FMK, resulting in a lack of GA fragmentation (FIG. 2d) and a 2-log reduction in numbers of infectious bacteria (FIG. 2e). Bacterial numbers decreased until 6 d p.i. suggesting a profound block in bacterial maturation. RNAi was then used to test the inhibitory effect of Z-WEHD-FMK on chlamydial replication in the absence of golgin-84. Interestingly, siRNA knockdown of golgin-84 restored chlamydial growth in host cells treated with Z-WEHD-FMK (FIG. 2e). To further support our hypothesis that golgin-84 can directly influence GA structure and Chlamydia replication, we generated an N-terminal deletion mutant (Δ218) of golgin-84 lacking potential protein interaction sites and inhibiting Chlamydia-induced fragmentation of the GA (FIG. 2f). Stable cell lines expressing either WT or Δ218 golgin-84 were generated by lentiviral transduction of golgin-84 KD cells. Numbers of infectious bacteria were reduced by ˜5 times in cells expressing Δ218 golgin-84 (FIG. 2g). Taken together, these data reveal that GA fragmentation in infected cells is a downstream event of golgin-84 cleavage and that Golgi fragmentation is a critical factor for efficient chlamydial growth.

We hypothesised that GA fragmentation before infection could boost bacterial replication. Therefore, GA fragmentation was induced by knockdown of giantin, Gpp130 (albeit less prominently) and golgin-84₁₁ (FIG. 3a). These siRNA and shRNA golgin-84 KD cells, plus luciferase KD control cells were then infected with C. trachomatis and the infectious progeny quantified at various times p.i. By 48 h p.i, up to 6 times more infectious bacteria were found in siRNA golgin-84 and giantin KD cells and up to 3 times more in Gpp130 KD cells (FIG. 3b), compared to control cells. In shRNA golgin-84 KD cells about 3 times more infectious bacteria could be recovered as early as 24 h p.i. compared to control cells, increasing to nearly 10 times more infectious bacteria at 40 h p.i. (FIG. 3c). Furthermore, treatment of infected cells at 8 h p.i. with a very low dose of nocodazole, still sufficient to induce GA fragmentation, enhanced bacterial propagation. Thus, GA fragmentation by various means boosts C. trachomatis reproduction. Interestingly, Salmonella enterica serovar typhimurium, a facultative intracellular bacterium, did not exhibit enhanced replication in golgin-84 KD cells, indicating a neutral function for golgin-84 in Salmonella infections. Based on the fact that no difference in numbers of inclusions in infected golgin-84 KD cells compared with control cells at low MOI was observed, we excluded the possibility that the growth stimulation elicited in golgin-84 shRNA cells was due to an increase in the primary infection. Consistent with the hypothesis of accelerated replication in golgin-84 KD cells, electron microscopy (EM) revealed the development of small, electron-dense particles, indicative of infectious bacteria (Ebs) as early as 24 h p.i. (FIG. 3d), but not in infected control cells (FIG. 3e). Taken together, these results support the notion that golgin-84 inactivation plays a decisive role in growth regulation of C. trachomatis, in that chlamydial maturation is dependent on and can be enhanced by depletion of golgin-84 throughout the replication cycle.

As GA structural alterations resulting from GM130 depletion are reported to cause an accumulation of improperly glycosylated proteins in the plasma membrane₂₂, we reasoned that Chlamydia-induced GA fragmentation could also interfere with processing of glycoproteins. As a marker of premature glycoproteins, proteins with terminal N-acetyl-D-glucosamine on the plasma membrane were detected using the GSA-II lectin from Griffonia simplicifolia₂₂. High levels of lectin binding were indicated by a bright staining of the plasma membrane in infected cells, whereas in uninfected cells no intense staining was observed (FIG. 4a). Although the GA still delivered glycoproteins to the cell surface, the normal processing of glycoproteins was altered in infected cells. Thus, C. trachomatis induced Golgi fragmentation affects the processing of glycoproteins in the GA.

Finally, we reasoned a breakdown of the Golgi structure into more, albeit smaller Golgi elements aligned around the inclusion may enhance lipid transport. Therefore, we treated infected cells with Z-WEHD-FMK, effectively preventing GA fragmentation, or with DMSO as a control and then labelled cells with fluorescent ceramide. Confocal images revealed ceramide was rapidly incorporated into the inclusion membrane within DMSO-treated cells and accumulated inside the inclusion in bacterial membranes (FIG. 4b). In contrast, Z-WEHD-FMK treated cells were only slightly fluorescent as the majority of lipid accumulated in a Golgi-like structure outside the inclusion (FIG. 4b). These observations clearly show that Golgi fragmentation enhances transport of sphingolipids to the bacterial inclusion at later time points during the infection.

Here we show infection of human epithelial cells with Chlamydia trachomatis induced a fragmentation of the GA triggered by the successive cleavage of golgin-84, which is affected by inhibitors of both inflammatory caspases and calpains. Yet, this does not exclude a role of bacterial proteases in golgin-84 processing. Truncation of golgin-84 resulted in the formation of Golgi ministacks around the bacterial inclusion. Inhibition of golgin-84 cleavage or expression of an inhibitory golgin-84 mutant (Δ218) prevented GA breakdown. Inhibition of GA fragmentation substantially reduced transport of lipids to the C. trachomatis inclusion and severely blocked bacterial propagation. Thus, golgin-84 turns out to be a crucial modulator of both structure and function of the GA during Chlamydia infection. Stable knockdown of golgin-84 (and also giantin) resulted in fully viable cells exhibiting a fragmented GA, leading to a substantial enhancement of chlamydial development.

Earlier work has indicated that sphingolipid acquisition begins as early as 2 h p.i., when internalized EBs have been converted into metabolically active Rbs^{2,3}. However, the requirement for sphingolipids is initially low and thought to increase dramatically with increased bacterial replication and expansion of the inclusion. This advanced stage of chlamydial expansion, starting at ˜20 h p.i., coincides with GA fragmentation. Interestingly, different molecular mechanisms of lipid transfer could account for the early and advanced lipid acquisition. For instance, whole Golgi derived vesicles could fuse with the inclusion membrane³. Alternatively, lipids could be transported individually to the inclusion membrane via specific lipid transporters or by pathways that bypass the GA^{23,24}. These transport mechanisms may be utilized in the early and/or advanced stages of chlamydial development.

Our discovery reveals a novel infectious mechanism by which an intracellular pathogen morphologically and functionally manipulates a host organelle to enhance lipid acquisition and secure its replication and development. Moreover, this work has identified novel molecular targets, including inflammatory caspases and calpains, which may potentially prove useful in the treatment of chlamydial infections.

Material and Methods

Reagents

Antibodies were obtained from the following sources: m-α-tubulin antibody (Sigma-Aldrich, Taufkirchen, Germany), m-α-golgin-84 (raised against the C-term), m-α-giantin, m-α-GM130, m-α-p230 (BD Biosciences, Heidelberg, Germany), m-α-Gpp130 (kind gift of Hans-Peter Hauri, Basel, Switzerland), m-α-chlamydia Hsp60 (Axxora, Lörrach, Germany), rb-α-LPS (Milan Analytica, La Roche, Switzerland). WEHD-FMK, DEVD-FMK and Caspase Inhibitor IV were purchased from R&D Systems (Wiesbaden-Nordenstadt, Germany), Calbiochem or Merck Biosciences (Darmstadt, Germany). BODIPY FL C5-Ceramide complexed to BSA and GS-II Alexa Fluor 594 was purchased from Invitrogen (Karlsruhe, Germany) and Hoechst 33342 was from Sigma-Aldrich (Seelze, Germany).

Infection and Quantification of Chlamydia Progeny

Chlamydia trachomatis serovar LGV L2, A and K and C. muridarum were propagated in HeLa cells in medium with 5% FCS at 35° C. in 5% CO₂. For infection, EBs were adsorbed to HeLa cells with a multiplicity from 0.5 to 5 and incubated for varying times, depending on assay. Numbers of chlamydial progeny were measured as the number of inclusion forming units (IFU) by counting inclusions on a fluorescence microscope using a 40× objective. IFUs were expressed as log IFU/ml.

Live Cell Imaging of Ceramide Transport

Cells were seeded onto glassbottom dishes (MatTek Corporation, Ashland, USA) and infected with C. trachomatis (MOI=2) or left uninfected for control. At indicated time points after infection, the dishes were transferred to an inverted confocal microscope (SP5, Leica, Bensheim, Germany) equipped with an incubator heated to 37° C. Every 30 sec, a Z-stack of 20 frames covering a depth of 10 μm was recorded for fluorescence and DIC using a resonant scanner. BODIPY FL C5-ceramide was added directly to the cells and bacterial nuclei were stained using Hoechst.

Live Cell Imaging of Infected EGFP-GM130 Expressing HeLa Cells

Cells were seeded into glass-like-bottom dishes (Ibidi, Munchen, Germany), transfected with a plasmid expressing human GM130 fused to EGFP (kind gift of M. A. De Matteis) using Lipofectamin 2000 (Invitrogen, Karlsruhe, Germany) according to the manufacturer's guidelines. The cells were subsequently either infected or mock-infected, and recorded 26 h p.i. at 37° C. on a Zeiss Axiovert 200M microscope with a Plan-Neofluar 100×/1.3 objective (Jena, Germany) over a period of 20 h. Every 3 minutes, a set of 2 images (phase contrast, GFP) was taken with a Hamamatsu Orca ER camera (Hamamatsu City, Japan). The system was controlled using Openlab software (Improvision, Coventry, UK), and individual frame overlays and videos were prepared using Volocity software (Improvision, Coventry, UK).

Ultrastructural Analysis of Cells by Electron Microscopy

Normal HeLa cells, golgin-84 KD cells and control KD cells were infected with C. trachomatis at a MOI of 2. Cells were fixed 24 h p.i. with 2.5% glutaraldehyde, post fixed with 1% OsO₄ for 45 min and contrasted with tannic acid and uranyl acetate. Specimens were dehydrated in a graduated ethanol series and embedded in PolyBed (Polysciences Europe GmbH, Eppelheim, Germany). After polymerisation, blocks were cut at 60-80 nm, contrasted with lead citrate and analysed in a LEO 906E TEM (Zeiss SMT, Oberkochen, Germany) equipped with a Morada camera (SIS, Münster, Germany). For quantification, micrographs of inclusions in the different KD cells were recorded at random; the size and electron density of particles were also scored. Particles larger than 3 μm with an electron, density comparable to that of the cytoplasm were regarded as reticular bodies, while smaller particles with higher electron density were counted as Chlamydia maturing to elementary bodies. A minimum of 350 particles per cell line were counted.

Treatment of Cells with Caspase Inhibitors

Caspase Inhibitor IV (40 μM), Z-WEHD-FMK (80 μM), Z-DEVD-FMK (80 μM), Calpeptin (25 μg/ml), Calpain Inhibitor III (100 μM), MG-132 (2.5 μM), PS1 (2.5 μM) and CA-074 Me (50 μM) was added to infected or mock-infected HeLa cells 9 h p.i (caspase inhibitors) or 8 h p.i. (calpain inhibitors). Cells were analysed 26 h p.i. and 28 h p.i. using immunofluorescence and immunoblotting. To test effects on C. trachomatis propagation, Z-WEHD-FMK was added 9 h p.i. to infected HeLa cells that were transfected with siRNA targeting luciferase or golgin-84. At 48 h p.i. Newly formed C. trachomatis bacteria were titrated on fresh HeLa cells.

siRNA Transfection

HeLa cells were seeded into 12-well plates, grown to 50-70% confluency, and transfected using RNAiFect (Qiagen) according to the manufacturer's guidelines. In brief, 1 μg of specific siRNA was added to EC-R buffer and incubated with 6 μl RNAiFect transfection reagent in a total volume of 100 μl. After 10-15 min the liposome/RNA mixture was added to the cells with 600 μl cell culture medium. After 1 day, cells were trypsinised and seeded into new cell culture plates depending on the experiments. Three days after transfection, the cells were infected and incubated as indicated above.

Determination of Cell Viability by WST-1 Assay

The viability of KD cells was determined using the WST-1 reagent (Roche, Mannheim, Germany). The assay was performed in triplicate using a 96-well format 3 days after transfection. The WST-1 reagent was diluted 1:5 in cell culture medium containing 5% FBS, added directly to the cultures and incubated for 4 h at 37° C. and 5% CO₂. As a negative control, untreated cells were lysed by Triton X-100 before addition of the reagent. The OD₄₅₀ was measured and absorption of untreated cells was set to 100%.

Immunofluorescence and Microscopy

Cells were seeded onto cover slips to visualise the Golgi apparatus in either infected or siRNA-transfected HeLa cells. At the indicated time points, cells were fixed with 2% PFA for 30 min at room temperature. Cells were then permeabilised with 0.2% Triton X-100/0.2% BSA in PBS for 30 min. Different Golgi markers were detected using specific antibodies, and bacteria were detected either using rb-α-LPS or m-α-Hsp60 antibodies followed by specific fluorescently labelled secondary antibodies and mounted in MOWIOL. Images were taken with a Leica TCS-SP (Wetzlar, Germany) confocal microscope and processed using Adobe Photoshop 6.0.

Immunoblotting

Infected or siRNA-transfected HeLa cells were lysed in RIPA buffer at the indicated time points. Protein concentrations were determined using the Pierce BCA kit (Perbio Science, Bonn, Germany), and 20 μg of total protein were analysed per lane on a reducing SDS-polyacrylamide gel. After separation, proteins were transferred onto a PVDF membrane by tank blotting. Specific antibodies were incubated with the membrane to detect antigens, followed by visualisation of the appropriate secondary antibodies using ECL reagent, as described previously (26).

Quantification of Golgi Fragments

Confocal images of specific samples were used to quantify Golgi fragmentation. The numbers of Golgi elements in 25 cells per experiment were counted after applying a fixed threshold to all images using the Analyse Particles function in ImageJ software. Three independent experiments were performed in duplicate to analyse fragmentation: upon infection, infection plus Z-WEHD-FMK treatment and staurosporine treatment.

Treatment with Staurosporine

To induce GA fragmentation by staurosporine, HeLa cells were incubated for 4 h at 35° C. with 2 μM staurosporine, and subsequently immunostained with the monoclonal mouse a-GM130 antibody.

siRNA

All siRNAs were designed and purchased from Qiagen (Hilden, Germany), and validated at the MPI for Infection Biology for their ability to knock-down mRNA expression of target genes by more than 70% in comparison to control cells transfected with siRNA for luciferase.

Validation of RNAi by q-PCR

siRNA validation was performed according to Machuy et al. 25. Briefly, 1 day prior to transfection 3,000 cells/well were seeded onto a 96-well plate. Transfection was performed with a final siRNA concentration of 56 nM with 0.25 μl RNAiFect (Qiagen) using luciferase targeting siRNA (target sequence in XYN₁₉ format: AACUUACGCUGAGUACUUCGA) as a control and the following target specific siRNAs:

| | |
|---|---|
| golgin-84 | (CTGAGTTTAGTGGTCCTAATA), |
| GM130 | (CAGGCTGGAGTTATACAAGAA), |
| golgin-245 | (CAGGAAATACATGAAATCCAA), |
| giantin | (AACTTCATGCGAAGGCCAAAT), |
| Gpp130 | (CAGGAGGACAATGTTGATGAA), |
| CASP | (CAGCGCCTGCACGATATTGAA). |

Knockdown measurements were performed independently 3 times. After 48 h, RNA was isolated using the RNeasy® 96 BioRobot® 8000 system (Qiagen). The relative amount of target mRNA was determined by q-PCR using the Quantitect™ SYBR® Green RT-PCR Kit following the manufacturer's instructions (Qiagen) and the following primers:

| | |
|---|---|
| GAPDH forward | 5′-GGTATCGTGGAAGGACTCATGAC-3′, |
| GAPDH reverse | 5′-ATGCCAGTGAGCTTCCCGTTCAG-3′, |
| golgin-84 forward | 5′-AATGCACCACGACCAACCA-3′, |
| golgin-84 reverse | 5′-AGGCAATTGGCCTTCTTGC-3′, |
| GM130 forward | 5′-AATATCAGCAGAGGAATAGCCCT-3′, |
| GM130 reverse | 5′-CAGCATTGTCCTTGGGTGTAT-3′, |
| golgin-245 forward | 5′-ATGTATATGCAACAACTGTGGGG-3′, |
| golgin-245 reverse | 5′-CGAGGTGAAGTAAACATCAGCC-3′, |
| giantin forward | 5′-CCCTAGACCCTGAATTACACCAA-3′, |
| giantin reverse | 5′-GGCAGAACAGTCCCTCCTTG-3′, |
| Gpp130 forward | 5′-CCCTCTCCGCCCAGTTACA-3′, |
| Gpp130 reverse | 5′-CTCCTCGTGTTGGCTTTTCA-3′, |
| CASP forward | 5′-AAAGACCAGCCTGAAAGTCGG-3′, |
| CASP reverse | 5′-CCAGGGATGAGCTGAAAAAGT-3′. |

The relative expression levels of target mRNA were normalised against control transfected cells. GAPDH was used as an internal standard.

Generation of Stable Golgin-84 KD Cell Lines

The golgin-84 shRNA construct oligonucleotides (Metabion, Martinsried, Germany) were annealed and ligated into the lentiviral vector pLVTHM targeting the following sequence in the 3′-UTR of golgin-84: GAGAACAGUGCACAAGAUUAU. Cells transduced with lentiviruses coding for a firefly luciferase shRNA (target sequence: AACUUACGCUGAGUACUUCGA) were used as a control. All constructs were verified by sequencing. Viruses carrying the shRNAs were produced by transfecting 293T cells with pLVTHM golgin84-15 or pLVTHM luci together with viral packaging vectors (psPAX2, pMD2G) by calcium phosphate transfection. Viruses were harvested from the supernatant 48 h post-transfection, filtrated through a 0.45 μm filter and applied to HeLa cells for lentiviral infection in the presence of polybrene (5 μg/ml). GFP positive cells were selected 6 d p.i., and single cells were transferred to a 96-well plate using FACS sorting. All vectors were obtained from D. Trono, Ecole Polytechnique Federale De Lausanne, Switzerland.

Generation of Stable Golgin-84 Expressing Cell Lines

The N-terminal truncated golgin-84 mutants were amplified from pENTR221-golga5 obtained from RZPD (Berlin, Germany) by Expand HighFidelity Taq (Roche, Mannheim, Germany) using specific primer pairs (Table S1). All primers were from MWG, Ebersberg, Germany. Products were cloned into pDONR221 (Invitrogen, Karlsruhe, Germany) by Clonase-II (Invitrogen, Karlsruhe, Germany) reaction and further subcloned into pLentiV5/DEST (Invitrogen, Karlsruhe, Germany). These vectors were used to generate specific lentiviruses, as described above. Golgin-84 KD #2 cells were infected with the respective virus and positive cells were selected using 10 μg/ml Blasticidin (Merck Biosciences (Darmstadt, Germany). Generated cell lines were passaged in the presence of 2 μg/ml Blasticidin. For infection experiments with C. trachomatis, Blasticidin was removed 1 day before infection. Plasmids and corresponding cell lines are listed in FIG. 5.

Lectin Binding

Binding of GS-II Alexa Fluor 594 has been described previously22. Briefly, 28 h p.i. C. trachomatis-infected or uninfected cells grown on cover slips were washed with icecold PBS++ (containing MgCl2 and CaCl2). 100 μg/ml fluorescent GS-II in PBS++ was added onto the cells and incubated for 2 h at 4° C. The cells were washed with PBS and then fixed with 2% PFA for 30 min at room temperature. Host cell and Chlamydia 24 DNA was visualised by Hoechst staining. Samples were analysed using an inverted confocal microscope (SP5, Leica, Bensheim, Germany).

Identification of Golgin-84 Cleavage Site by MS

Full-length human golgin-84 was amplified from pENTR221-golga5 by Expand HighFidelity Taq (Roche, Mannheim, Germany) using specific primer pairs that contained a C-terminal myc-tag 5′-AAAAAGCAGGCTGAACCATGTCTTGGTTTGTTGATCTTGC-3′ and 5′-AGAAAGCTGGGTATCACTACAGATCTTCTTCAGAAATAAGTTTTTGTTCT TTGCCATATGGTTGGTCGTGGTGC-3′. Products were cloned into pDONR221 (Invitrogen, Karlsruhe, Germany) by Clonase-II (Invitrogen, Karlsruhe, Germany) reaction and further subcloned into pDest760. Tagged golgin-84 was transiently expressed and cells were infected with C. trachomatis. 36 h p.i. cells were lysed in RIPA buffer containing protease inhibitor cocktail (Roche, Mannheim Germany) and tagged golgin-84 was immunoprecipitated by anti-myc antibodies 9E10 followed by Dynabeads Protein G (Invitrogen, Karlsruhe, Germany). Precipitated golgin-84 was subjected to SDS-PAGE and the gel was stained with Serva DensiStain Blue G (Serva, Electrophoresis GmbH, Heidelberg, Germany). The 65 kDa golgin-84 cleavage product was cut from the gel and digested with modified trypsin (Promega, Mannheim, Germany) according to the supplier's instructions. Eluted peptides were analysed by Nano-LC-MS/MS (Nano-Acquity, Waters, Milford, Mass., USA, combined with a LTQ-Orbitrap, Thermo Fisher, Waltham, Mass., USA). A linear gradient from water to acetonitrile (both with 0.1% formic acid) was used to elute the peptides with a flow of 200 nl/min from a self-prepared Nano-RP-column (Reprosil-Pur 300 C18, 3 um, Dr. Maisch, Ammerbuch-Entringen, Germany, packed in PicoTip Emitter, 75 um×150 mm, New Objective, Woburn, Mass., USA). Throughout the whole run, MS and MS/MS spectra were collected in data-dependent acquisition mode. For protein identification, an InHouse version of MASCOT-server (MatrixScience, London, UK) was used to search against the Swiss-Prot database (Release 55.4).

### REFERENCE LIST

1. R. A. Carabeo, D. J. Mead, and T. Hackstadt, “Golgi-dependent transport of cholesterol to the Chlamydia trachomatis inclusion,” Proc. Natl. Acad. Sci. U.S.A 100(11), 6771 (2003).
2. T. Hackstadt, D. D. Rockey, R. A. Heinzen, and M. A. Scidmore, “Chlamydia trachomatis interrupts an exocytic pathway to acquire endogenously synthesized sphingomyelin in transit from the Golgi apparatus to the plasma membrane,” EMBO J. 15(5), 964 (1996).
3. M. A. Scidmore, E. R. Fischer, and T. Hackstadt, “Sphingolipids and glycoproteins are differentially trafficked to the Chlamydia trachomatis inclusion,” J. Cell Biol. 134(2), 363 (1996).
4. G. M. Hatch and G. McClarty, “Phospholipid composition of purified Chlamydia trachomatis mimics that of the eucaryotic host cell,” Infect. Immun. 66(8), 3727 (1998).
5. C. van Ooij, et al., “Host cell-derived sphingolipids are required for the intracellular growth of Chlamydia trachomatis,” Cell Microbiol. 2(6), 627 (2000).
6. J. L. Wylie, G. M. Hatch, and G. McClarty, “Host cell phospholipids are trafficked to and then modified by Chlamydia trachomatis,” J Bacteriol. 179(23), 7233 (1997).
7. M. A. De Matteis and A. Luini, “Exiting the Golgi complex,” Nat. Rev. Mol. Cell Biol. 9(4), 273 (2008).
8. J. Shorter and G. Warren, “Golgi architecture and inheritance,” Annu. Rev. Cell Dev. Biol. 18, 379 (2002).
9. B. Short, A. Haas, and F. A. Barr, “Golgins and GTPases, giving identity and structure to the Golgi apparatus,” Biochim. Biophys. Acta 1744(3), 383 (2005).
10. N. Altan-Bonnet, et al., “Golgi inheritance in mammalian cells is mediated through endoplasmic reticulum export activities,” Mol. Biol. Cell 17(2), 990 (2006).
11. A. Diao, et al., “The coiled-coil membrane protein golgin-84 is a novel rab effector required for Golgi ribbon formation,” J. Cell Biol. 160(2), 201 (2003).
12. M. Lowe, et al., “Cdc2 kinase directly phosphorylates the cis-Golgi matrix protein GM130 and is required for Golgi fragmentation in mitosis,” Cell 94(6), 783 (1998).
13. Y. Wang, et al., “A direct role for GRASP65 as a mitotically regulated Golgi stacking factor,” EMBO J. 22(13), 3279 (2003).
14. R. Chiu, L. Novikov, S. Mukherjee, and D. Shields, “A caspase cleavage fragment of p115 induces fragmentation of the Golgi apparatus and apoptosis,” J. Cell Biol. 159(4), 637 (2002).
15. M. Lowe, J. D. Lane, P. G. Woodman, and V. J. Allan, “Caspase-mediated cleavage of syntaxin 5 and giantin accompanies inhibition of secretory traffic during apoptosis,” J. Cell Sci. 117(Pt 7), 1139 (2004).
16. M. Mancini, et al., “Caspase-2 is localized at the Golgi complex and cleaves golgin-160 during apoptosis,” J. Cell Biol. 149(3), 603 (2000).
17. K. Rajalingam, et al., “Epithelial cells infected with Chlamydophila pneumoniae (Chlamydia pneumoniae) are resistant to apoptosis,” Infect. Immun. 69(12), 7880 (2001).
18. T. Fan, et al., “Inhibition of apoptosis in chlamydia-infected cells: blockade of mitochondrial cytochrome c release and caspase activation,” J. Exp. Med. 187(4), 487 (1998).
19. J. L. Hood, W. H. Brooks, and T. L. Roszman, “Differential compartmentalization of the calpain/calpastatin network with the endoplasmic reticulum and Golgi apparatus,” J. Biol. Chem. 279(41), 43126 (2004).
20. D. E. Goll, et al., “The calpain system,” Physiol Rev. 83(3), 731 (2003).
21. K. Suzuki and H. Sorimachi, “A novel aspect of calpain activation,” FEBS Lett. 433(1-2), 1 (1998).
22. M. A. Puthenveedu, et al., “GM130 and GRASP65-dependent lateral cisternal fusion allows uniform Golgi-enzyme distribution,” Nat. Cell Biol. 8(3), 238 (2006).
23. J. C. Holthuis and T. P. Levine, “Lipid traffic: floppy drives and a superhighway,” Nat. Rev. Mol. Cell Biol. 6(3), 209 (2005).
24. M. Marie, R. Sannerud, Dale H. Avsnes, and J. Saraste, “Membrane traffic in the secretory pathway: Take the ‘A’ train: on fast tracks to the cell surface,” Cell Mol. Life Sci. 65(18), 2859 (2008).
25. N. Machuy, et al., “A global approach combining proteome analysis and phenotypic screening with RNA interference yields novel apoptosis regulators,” Mol. Cell Proteomics. 4(1), 44 (20
26. D. Heuer, V. Brinkmann, T. F. Meyer, and A. J. Szczepek, “Expression and translocation of chlamydial protease during acute and persistent infection of the epithelial HEp-2 cells with Chlamydophila (Chlamydia) pneumoniae,” Cell Microbiol. 5(5), 315 (2003).
27. S. Resnikoff et al., Bull. World Health Organ 82, 844-851 (2004).
28. A. C. Gerbase, J. T. Rowley, T. E. Mertens, Lancet 351 Suppl 3, 2-4 (1998).
29. S. P. Salcedo and D. W. Holden, Curr. Opin. Microbiol. 8, 92-98 (2005).
30. S. S. Grieshaber, N. A. Grieshaber, T. Hackstadt, Molecular Biology of the Cell 13, 404A (2002).
31. S. P. Salcedo and D. W. Holden, EMBO J. 22, 5003-5014 (2003).
32. T. Hackstadt, D. D. Rockey, M. A. Scidmore, R. A. Heinzen, Molecular Biology of the Cell 6, 1340 (1995).
33. Allan L, and Pearce J H. 1983. Differential amino acid utilization by Chlamydia psittaci (strain guinea pig inclusion conjunctivitis) and its regulatory effect on chlamydial growth. J Gen Microbiol 129:1991-2000.
34. Al-Younes H M, Rudel T, Brinkmann V, Szczepek A J, and Meyer T F. 2001. Low iron availability modulates the course of Chlamydia pneumoniae infection. Cell Microbiol 3:427-437.
35. Anttila T, Saikku P, Koskela P, Bloigu A, Dillner J, Ikaheimo I, Jellum E, Lehtinen M, Lenner P, Hakulinen T, Narvanen A, Pukkala E, Thoresen S, Youngman L, and Paavonen J. 2001. Serotypes of Chlamydia trachomatis and risk for development of cervical squamous cell carcinoma. JAMA 285:47-51.
36. Balin B J, Gerard H C, Arking E J, Appelt D M, Branigan P J, Abrams J T, Whittum-Hudson J A, and Hudson A P. 1998. Identification and localization of Chlamydia pneumoniae in the Alzheimer's brain. Med Microbiol Immunol (Berl) 187:23-42.
37. Beatty W L, Byrne G I, and Morrison R P. 1994. Repeated and persistent infection with Chlamydia and the development of chronic inflammation and disease. Trends Microbiol 2:94-98.
38. Blasi F, Arosio C, and Cosentini R. 1999. Chlamydia pneumoniae: Epidemiology. In: Chlamydia pneumoniae, the lung and the heart. Allegra L, and Blasi F (Eds). Springer-Verlag, Milano, Italy. pp. 52-61.
39. Braunwald E. 1997. Shattuck lecture—cardiovascular medicine at the turn of the millennium: triumphs, concerns, and opportunities. N Engl J Med 337:1360-1369.
40. Campbell L A and Kuo C C (2003). Chlamydia pneumoniae and atherosclerosis Semin. Respir. Infect. 18, 48-54.
41. Chesson H W, and Pinkerton S D. 2000. Sexually transmitted diseases and the increased risk for HIV transmission: implications for cost-effectiveness analyses of sexually transmitted disease prevention interventions. J Acquir Immune Defic Syndr 24:48-56.
42. de Boer O J, van der Wal A C, and Becker A E. 2000. Atherosclerosis, inflammation, and infection. J Pathol 190:237-243.
43. Erntell M, Ljunggren K, Gadd T, and Persson K. 1989. Erythema nodosum—a manifestation of Chlamydia pneumoniae (strain TWAR) infection. Scand J Infect Dis 21:693-696.
44. Everett K D E, Bush R M, and Andersen A A. 1999. Emended description of the order Chlamydiales, proposal of Parachlamydiaceae fam. nov. and Simkaniaceae fam. nov., each containing one monotypic genus, revised taxonomy of the family Chlamydiaceae, including a new genus and five new species, and standards for the identification of organisms. Int J Syst Bacteriol 49:415-440.
45. Grayston J T, Campbell L A, Kuo C C, Mordhorst C H, Saikku P, Thom D H, and Wang S R 1990. new respiratory tract pathogen: Chlamydia pneumoniae strain TWAR. J Infect Dis 161:618-625.
46. Guaschino S, and De Seta F. 2000. Update on Chlamydia trachomatis. Ann N Y Acad Sci 900:293-300.
47. Gupta S, and Camm A J. 1998. Chlamydia pneumoniae, antimicrobial therapy and coronary heart disease: a critical overview. Coron Artery Dis 9:339-343.
48. Haidl S, Ivarsson S, Bjerre I, and Persson K. 1992. Guillain-Barré syndrome after Chlamydia pneumoniae infection. N Engl J Med 326:576-577.
49. Hammerschlag M R. 2002. The intracellular life of chlamydiae. Semin Pediatr Infect Dis 13:239-248.
50. Kaltenboeck B, Kousoulas K G, and Storz J. 1993. Structures of and allelic diversity and relationships among the major outer membrane protein (ompA) genes of the four chlamydial species. J Bacteriol 175:487-502.
51. Koskiniemi M, Gencay M, Salonen O, Puolakkainen M, Farkkila M, Saikku P, and Vaheri A. 1996. Chlamydia pneumoniae associated with central nervous system infections. Eur Neurol 36:160-163.
52. Laurila A L, Anttila T, Laara E, Bloigu A, Virtamo J, Albanes D, Leinonen M, and Saikku P. 1997. Serological evidence of an association between Chlamydia pneumoniae infection and lung cancer. Int J Cancer 74:31-34.
53. Moulder J W. 1991. Interaction of chlamydiae and host cells in vitro. Microbiol Rev 55:143-190.
54. Ramirez J A. 1996. Isolation of Chlamydia pneumoniae from the coronary artery of a patient with coronary atherosclerosis. The Chlamydia pneumoniae/Atherosclerosis Study Group. Ann Intern Med 125:979-982.
55. Schachter J. 1999. Infection and disease epidemiology. In: Chlamydia: intracellular biology, pathogenesis, and immunity. Stephens R S (ed), American Society for Microbiology, Washington, D.C. pp. 139-169.
56. Stephens L C, McChesney A E, and Nockels C F. 1979. Improved recovery of vitamin E-treated lambs that have been experimentally infected with intratracheal Chlamydia. Br Vet j 135: 291-293.
57. Villareal C, Whittum-Hudson J A, and Hudson A P. 2002. Persistent Chlamydiae and chronic arthritis. Arthritis Res 4:5-9.

1. A screening method for identification of a compound suitable for treatment, prevention or/and diagnosis of an infection with microorganisms from the family Chlamydiaceae, comprising identification of an calpain inhibitor.

2. The screening method of claim 1 comprising the steps
(a) providing calpain,
(b) contacting a compound with calpain,
(c) determining if the compound of (b) inhibits calpain activity, and
(d) selecting a compound which inhibits calpain activity.

3. The method of claim 1, wherein the calpain is provided in a cell or cell extract or as isolated protein.

4. The method of claim 1, wherein calpain is calpain-2 and the calpain inhibitor is a calpain-2 inhibitor.

5. A screening method for identification of a compound suitable for treatment, prevention or/and diagnosis of an infection with microorganisms from the family Chlamydiaceae, comprising identification of a compound capable of inhibiting cleavage of a Golgi protein.

6. The screening method of claim 5 comprising the steps
(a) providing a Golgi protein,
(b) contacting a compound with a Golgi protein,
(c) determining if the compound of (b) inhibits Golgi protein cleavage, and
(d) selecting a compound which inhibits Golgi protein cleavage.

7. The screening method of claim 5, wherein the Golgi protein is selected from the group consisting of GASP, p115, GRASP55, GRASP65, golgin-45, Bicaudal D1 and D2, golgin-245, GMAp210, Rud3p, GM130, Gpp130, golgin-84, golgin-160, preferably the Golgi protein is selected from the group consisting of Gpp130, golgin-84, golgin-160, giantin, and more preferably the Golgi protein is golgin-84 or golgin-160.

8. The method of claim 5, wherein cleavage of the Golgi protein by calpain is inhibited.

9. The method of claim 8, wherein calpain is calpain-2 and the compound suitable for treatment, prevention or/and diagnosis of an infection with microorganisms from the family Chlamydiaceae is a calpain-2 inhibitor.

10. The method of claim 5, wherein the Golgi protein is provided in a cell or cell extract or as isolated protein.

11. The method of claim 10, wherein the cell is infected with microorganisms from the family Chlamydiaceae.

12. A screening method for identification of a compound suitable for treatment, prevention or/and diagnosis of an infection with microorganisms from the family Chlamydiaceae, comprising identification of a compound capable of inhibiting Golgi apparatus fragmentation in a cell infected with microorganisms from the family Chlamydiaceae.

13. The screening method of claim 12 comprising the steps
(a) providing a cell comprising a Golgi apparatus, which cell is capable of being infected with microorganisms from the family Chlamydiaceae,
(b) contacting the cell with a compound and with microorganisms from the family Chlamydiaceae,
(c) determining if the compound inhibits Golgi apparatus fragmentation in the cell, and
(d) selecting a compound which inhibits Golgi apparatus fragmentation in the cell.

14. The method of claim 12, wherein calpain-dependent Golgi apparatus fragmentation is inhibited.

15. The method of claim 12, wherein calpain is calpain-2 and the compound suitable for treatment, prevention or/and diagnosis of an infection with microorganisms from the family Chlamydiaceae is a calpain-2 inhibitor.

16. The method of claim 12, wherein Golgi apparatus fragmentation depending upon cleavage of a Golgi protein is inhibited.

17. The method of claim 12, wherein the Golgi protein is selected from the group consisting of GASP, p115, GRASP55, GRASP65, golgin-45, Bicaudal D1 and D2, golgin-245, GMAp210, Rud3p, GM130, Gpp130, golgin-84, golgin-160, preferably the Golgi protein is selected from the group consisting of Gpp130, golgin-84, golgin-160, giantin, and more preferably the Golgi protein is golgin-84 or golgin-160.

18. A pharmaceutical composition comprising as an active ingredient an inhibitor of calpain, an inhibitor of cleavage of a Golgi protein, or/and an inhibitor of Golgi apparatus fragmentation, optionally together with suitable carriers, diluents, adjuvants or/and auxiliary substances.

19. The pharmaceutical composition of claim 18 suitable for treatment, prevention or/and diagnosis of an infection with microorganisms from the family Chlamydiaceae.

20. The pharmaceutical composition of claim 18, wherein the pharmaceutical composition is for use in human or/and veterinary medicine.

21. The pharmaceutical composition of claim 18 comprising at least one further active ingredient for the prevention or/and treatment of infections with microorganisms from the family Chlamydiaceae.

22. The pharmaceutical composition of claim 18, wherein the at least one further active ingredient is selected from antibiotics such as macrolides, quinolones and combinations thereof.

23. The pharmaceutical composition of claim 18 for the treatment of patients with chronic infections with microorganisms from the family Chlamydiaceae.

24. The pharmaceutical composition of claim 18, wherein the calpain inhibitor is a calpain-2 inhibitor.

25. A method for treatment or/and prevention of an infection with microorganisms from the family Chlamydiaceae, the method comprising the administration of an inhibitor of calpain, an inhibitor of cleavage of a Golgi protein, or/and an inhibitor of Golgi apparatus fragmentation, in a amount effective in therapy or/and prevention to a subject in need thereof.

26. The method of claim 25, further comprising administering at least one further active ingredient for the prevention or/and treatment of an infection with microorganisms from the family Chlamydiaceae in a amount effective in therapy or/and prevention.

27. The method of claim 25, wherein the calpain inhibitor is a calpain-2 inhibitor.

28. A method of diagnosing an infection with a microorganism from the family Chlamydiaceae, comprising determination of a Golgi protein, Golgi apparatus fragmentation or/and calpain in a biological sample.

29. The method of claim 28, wherein calpain is calpain-2.
